SEQUENCE LISTING (2 sequences)

SEQ ID NO: 1 - DNA, 2294 bp, Chicken anemia virus
gcattccgagtggttactattccatcaccattctagcctgtacacagaaagtcaagatggacgaatcgctcgacttcgctcgcgattcgtcgaaggcggggggccggaggccccccggtggcccccctccaacgagtggagcatgtacaggggggtacgtcatcagtacaggggggtacgtcacaaaaaggcgttcccgtacaggggggtacgtcacgcgtacaggggggtacgtcacagccaatcagaagctgccacgttgcgaaagtgacgtttcgaaaatgggcggcgcaagcctctctatatattgagcgcacataccggtcggcagtaggtatacgcaaggcggtccgggtggatgcacgggaacgacggacaaccggccgctgggggcagtgaatcggcgcttagccgagaggggcagcctgggcccagcggagccgcgcaggggcaagtaatttcaaatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaagaaggtgtataagactgtaagatggcaagacgagctcgcagaccgagaggccgattttacgccttcagaagaggacggtggcaccacctcaagcgacttcgacgaagatataaatttcgacatcggaggagacagcggtatcgtagacgagcttttaggaaggcctttcacaacccccgccccggtacgtatagtgtgaggctgccgaacccccaatctaccatgactatccgcttccaaggagtcatctttctcacagaaggactcattctgcctaaaaacagcacagcggggggctatgcagaccacatgtacggggcgagagtcgccaagatctctgtgaacctgaaagagttcctgctagcgtcaatgaacctgacatacgtgagcaaaatcggaggccccatcgccggtgagttgattgcggacgggtctaaatcacaagccgcggagaactggcctaattgctggctgccgctagataataacgtgccctccgcgacaccatcggcatggtggagatgggccttaatgatgatgcagcccacggactcttgccggttctttaatcaccctaagcagatgaccctgcaagacatgggtcgcatgtttgggggctggcacctattccgacacattgaaacccgctttcagctccttgccactaagaatgagggatccttcagccccgtggcgagtcttctctcccagggagagtacctcacgcgtcgggacgatgttaaatacagcagcgatcaccagaaccggtggcgaaaaggcgaacaaccgatgacggggggtattgcttatgcgaccgggaaaatgagacccgacgagcaacagtaccctgctatgcccccagaccccccgatcatcaccagtactacagcgcaaggcacgcaagtccgctgcatgaatagcacgcaagcttggtggtcatgggacacatatatgagctttgcaacactcacagcactcggtgcacaatggtcttttcctccagggcaacgttcagtttctagacggtccttcaaccaccacaaggcgagaggagccggggaccccaaaggccagagatggcacaccctggtgccgctcggcacggagaccatcaccgacagctacatgggagcacccgcatcagagctggacacaaatttctttacgctttacgtagcgcaaggtacaaataagtcgcagcagtacaagttcggcacagctacatacgcgctaaaggagccggtaatgaagagcgattcatgggcagtggtacgcgtccagtcggtctggcaactgggtaacaggcagagaccatacccatgggacgtcaactgggccaacagcaccatgtactggggcgggcagccctgaaaagggggggggctaaagcccccccttgaacccccccctgggggggattcccccccagaccccccctttaaatagcactcaataaacgcagcaattggctttatcgcacaatc

SEQ ID NO: 2 - protein, 786 aa, Chicken anemia virus
MARRARRPRGRFYAFRRGRWHHLKRLRRRYKFRHRRRQRYRRRAFRKAFHNPRPGTYSVRLPNPQSTMTIRFQGVIFLTEGLILPKNSTAGGYADHMYGARVAKISVNLKEFLLASMNLTYVSKIGGPIAGELIADGSKSQAAENWPNCWLPLDNNVPSATPSAWWRWALMMMQPTDSCRFFNHPKQMTLQDMGRMFGGWHLFRHIETRFQLLATKNEGSFSPVASLLSQGEYLTRRDDVKYSSDHQNRWRKGEQPMTGGIAYATGKMRPDEQQYPAMPPDPPIITSTTAQGTQVRCMNSTQAWWSWDTYMSFATLTALGAQWSFPPGQRSVSRRSFNHHKARGAGDPKGQRWHTLVPLGTETITDSYMGAPASELDTNFFTLYVAQGTNKSQQYKFGTATYALKEPVMKSDSWAVVRVQSVWQLGNRQRPYPWDVNWANSTMYWGGQPMHGNDGQPAAGGSESALSREGQPGPSGAAQGQVISNERSPRRYSTRTINGVQATNKFTAVGNPSLQRDPDWYRWNYNHSIAVWLRECSRSHAKICNCGQFRKHWFQECAGLEDRSTQASLEEAILRPLRVQGKRAKRKLDYHYSQPTPNRKKVYKTVRWQDELADREADFTPSEEDGGTTSSDFDEDINFDIGGDSGIVDELLGRPFTTPAPVRIVMNALQEDTPPGPSTVFRPPTSSRPLETPHCREIRIGIAGITITLSLCGCANARAPTLRSATADNSESTGFKNVPDLRTDQPKPPSKKRSCDPSEYRVSELKESLITTTPSRPRTARRCIRL

(19) United States
(12) Patent Application Publication
(10) Pub. No.: US 2008/0057078 A1
(43) Pub. Date: Mar. 6, 2008

(54) Chicken Anemia Virus Vaccine from Cell Line

(76) Inventor: Joan D. Leonard, Lenexa, KS (US)

Correspondence Address: NEEDLE & ROSENBERG, P.C., SUITE 1000, 999 PEACHTREE STREET, ATLANTA, GA, 30309-3915, US

(21) Appl. No.: 10/591,725

(22) PCT Filed: Mar. 7, 2005
(86) PCT No.: PCT/US05/07148

§ 371 Date: May 7, 2007

Related U.S. Application Data
(60) Provisional application No. 60/550,573, filed on Mar. 5, 2004.

(51) Int. Cl. A61K 39/00; A61P 43/00

(52) U.S. Cl. 424/204.1

(57) ABSTRACT

Provided is a chicken infectious anemia virus (CIAV) vaccine, comprising live CIAV passaged in MDCC-MSB-1 (MSB-1) cells, wherein the vaccine does not cause Marek's Disease. Also provided is a CIAV vaccine comprising a CIA virus having the sequence of SEQ ID NO: 1. A method of making a CIAV vaccine is provided, comprising culturing CIAV in MSB-1 cells, and removing or killing any Marek's disease virus present in the CIAV-containing MSB- 1 culture. Provided a method of immunizing a chicken against CIAV infection, comprising administering to the chicken an amount of the CIAV vaccine of the invention sufficient to induce an immune response to CIAV.

### BACKGROUND OF THE INVENTION

1. Field of the Invention

The invention relates generally to a vaccine for chicken infectious anemia virus, methods of making the vaccine and methods of immunization using the vaccine.

2. Background

CIAV causes clinical and subclinical disease in chickens, and is recognized as an important avian pathogen worldwide. In young chickens, CIAV causes a transient severe anemia due to destruction of erythroblastoid cells in the bone marrow and immunodeficiency due to depletion of cortical thymocytes. The depletion of cortical thymocytes is considered to cause a transient immunodeficiency resulting in enhanced concurrent infections and to vaccination failures. The depletion of thymocytes and most likely also of erythroblastoid cells occurs via VIAC-induced apoptosis.

CIAV is a small virus of a unique type with a particle diameter of 23-25 mn and a genome consisting of a circular single-stranded (minus strand) DNA. This DNA multiplies in infected cells via a circular double-stranded replicative intermediate. CIAV is not related to other known animal single stranded circular DNA viruses, such as porcine circovirus and psittacine beak-and-feather disease virus.

The major transcript from the CIAV genome is an unspliced polycistronic mRNA of about 2100 nucleotides encoding three proteins of 51.6 kDa (VP1), 24.0 kDa (VP2) and 13.6 kDa (VP3 or apoptin). All three proteins are synthesized in CAIV-infected cells.

To reduce the economic damage caused by CIAV infection, it is necessary to provide a cost-effective vaccine against CIAV. Prior attempts to provide a CIAV vaccine have required the passaging and propagation of CIAV in CIAV-susceptible SPF-embryos (See Vielitz and Voss, International Symposium on Infectious Bursal Disease and Chicken Infectious Anemia, Rauischholzhausen, Germany, 21-24 June 19114). Attempts to produce CIAV in cell lines have been problematic due to infection of susceptible cell lines with Marek's disease virus. Thus, a need exists for a vaccine produced ill cultured cells that will not cause Marek's disease.

The present invention meets the needs of this field by providing a vaccine without the disadvantages of embryo passaging and without the disadvantages of Marek's disease virus contamination.

### SUMMARY OF THE INVENTION

In accordance with the purpose(s) of this invention, as embodied and broadly described herein, this invention, in one aspect, relates to a chicken infectious anemia virus (CIAV) vaccine, comprising live CIAV passaged in MDCC-MSB-1 (MSB-1) cells, wherein the vaccine does not cause Marek's Disease.

In another aspect, the invention provides a CIAV vaccine comprising a CIA virus having the sequence of SEQ ID NO: 1.

In another aspect, the invention provides a method of making a CIAV vaccine, comprising culturing CIAV in MSB-1 cells, and removing or killing any Marek's disease virus present in the CIAV-containing MSB-1 culture. The method can include subjecting the CIAV-containing MSB-1 cell culture to at least 3 cycles of freezing and thawing, followed by a step of maintaining the cells for about 3 days at about 37° C. Alternatively, filtration may be used, or centrifugation followed by treatment at about 37° C.

In a further aspect, the invention provides a method of immunizing a chicken against CIAV infection, comprising administering to the chicken an amount of the CIAV vaccine of the invention sufficient to induce an immune response to CIAV.

The invention has the advantage that it provides a CIAV vaccine that can be produced in a cell line and is free of contaminating viruses.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows PCR products (l=marker, 2-Del Ros, 3=Intervet CIAV embryo adapted and attenuated vaccine, 4=1:2 cells, 5=1:2 supernatant, 6=1:10 cells, 7=1:10 supernatant, 8=MSB-1 cells only).

FIG. 2 shows restriction enzyme analysis with HindIII (1=marker, 2=CIAV Del Ros uncut, 3=CIAV Del Ros HindHIII, 4=Intervet CIAV uncut, 5=Intervet CIAV HindIII, 6=1:2 Intervet CIAV uncut, 7=1:2 Intervet CIAV sample HindIII, 8=1:10 Intervet CIAV HindIII).

FIG. 3 shows the effect of freeze-thaw on the viability of MDV (Rispen's virus).

FIG. 4 shows the effect of 37° C. on the viability of MDV (Rispen's virus) after 3 freeze-thaw cycles.

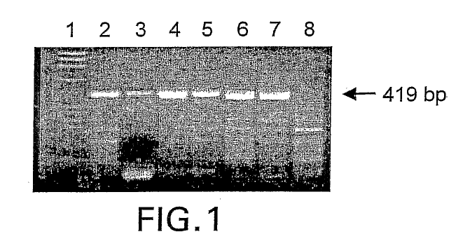

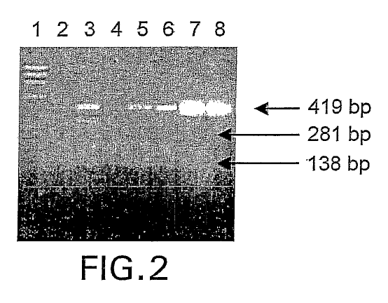

### DETAILED DESCRIPTION OF THE INVENTION

The present invention may be understood more readily by reference to the following detailed description of preferred embodiments of the invention and the Examples included therein and to the Figures and their previous and following description.

As used in the specification and the appended claims, the singular forms “a,” “an” and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about” or “approximately,” it will be understood that the particular value forms another embodiment. It will be further understood that the endpoints of each of the ranges are significant both in relation to the other endpoint, and independently of the other endpoint.

The invention provides a chicken infectious anemia virus (CIAV) vaccine, comprising live CIAV passaged in MDCC-MSB-1 (MSB-1) cells, wherein the vaccine does not cause Marek's Disease.

The CIAV vaccine of the invention does not produce gross lesions in a significant number of chicken embryos. The vaccine has been tested in embryos, and in the studies done, produces lesions in fewer than 10% of embryos. This is in contrast to a different CIAV vaccine that is produced in chicken embryos, and causes significant lesions in the embryos.

The CIAV vaccine of the invention also does not produce significant anemia in chicken embryos.

The invention provides a CIAV vaccine comprising of any of the reported strains (e.g., intervet strain, Cux-1 strain, Texas strain, DRP5 (Del Ros after 5 passages), CAV-15 strain, etc.). For example, invention provides a CIAV vaccine comprising a CIAV having the sequence of SEQ ID NO: 1. This is the sequence the Del Ros strain. The invention also provides a CIAV vaccine comprising any CIAV strain that is newly isolated or is a modified form of a known strain.

A method of making a CIAV vaccine is provided, comprising culturing CIAV in MSB-1. In addition to providing a method of making MSB-1-cultured CIAV free of Marek's disease virus (MDV) (see below and Example 1), the method can also produce CIAV to a titer of at least 10^{8.1}. This is a higher titer than is typically obtained for this virus in MSB-1 cells. The details of one example of this process are provided in Example 1. It is recognized that other methods for culturing CIAV in MSB-1 cells may be routinely developed and practiced.

The method of making a CIAV vaccine can be used with any of the reported CIAV strains (e.g., intervet strain, Cux-1 strain, Texas strain, DRP5 (Del Ros after 5 passages), CAV-15 strain, etc.). For example, the method of making a CIAV vaccine can use a CIAV having the sequence of SEQ ID NO: 1. The method of making a CIAV vaccine can also use any CIAV strain that is newly isolated or is a modified form of a known strain.

The method of making a CIAV vaccine can farther comprise the step of separating the cultured CIAV from the MSB-1 cells, which typically contain MDV. CIAV is secreted into the culture medium, thus allowing for a variety steps for separating the CIAV from MSB-1 cells. For example, the method of making a CIAV vaccine can comprise a step of subjecting the CIAV to at least 3 cycles of freezing and thawing. This disrupts the cells and inactivates a substantial amount of the MDV (an obligate intracellular pathogen). This step is usually followed with a step of maintaining the cells for about 3 days at about 37° C. This inactivates any remaining MDV. A further method of making the CIAV grown in MSB-1 cells free of MDV can comprise the step of filtering the virus-containing MSB-1 cells through a 5 micron filter. Filtering can rupture the cells because they are fragile, and it also removes any intact cells. Examples of these processes for removing MDV from the CIAV vaccine and for killing any MDV in the CIAV culture are provided in Example 1 and Example 9. It is recognized that other methods for obtaining the CIAV vaccine from MSB-1 cells that is free of MDV may be routinely developed and practiced. For example, a process of centrifuging the CIAV infected MSB-1 cells to remove cells and most of the MDV, followed by cycles of freeze-thaw of the supernatant and maintenance at 37° C. to kill any remaining MDV is also effective. Thus the methods of making the CIAV vaccine provided herein produce a vaccine that does not cause Marek's disease in chickens immunized with the vaccine.

The invention provides a method of immunizing a chicken against CIAV infection, comprising administering to the chicken an amount of the CIAV vaccine of the invention sufficient to induce an immune response to CIAV. The immune response produced is protective against infection by CIAV. Thus, the immune response is also protective against clinical disease caused by CIAV infection. Although, in one example, the present CIAV vaccine is not attenuated immunized chickens (e.g., embryos, chicks and hens) do not typically get sick, because of the recognized resistance to this virus.

The term “inactivated,” also referred to as “killed,” means that the CIAV virus is treated by any of several means known to the art so that they no longer grow or reproduce, but that the microorganisms are still capable of eliciting an immune response in the target animal. Examples of inactivating agents are: formalin, azide, freeze-thaw, sonication, heat treatment, sudden pressure drop, detergent (especially non-ionic detergents), lysozyme, phenol, proteolytic enzymes, propiolactone, Thimerosal (see U.S. Pat. No. 5,338,543 Fitzgerald, et al.), and binary ethyleneimine (see U.S. Pat. No. 5,565,205 Petersen, et al.).

Alternatively, the CIAV vaccine can be attenuated. The term “attenuated,” also referred to as “modified live,” is intended to refer to living CIAV which has been attenuated (modified) by any of a number of methods known in the art including, but not limited to, multiple serial passage, temperature sensitive attenuation, mutation, or the like such that the resultant strain is relatively non-pathogenic to an avian species. The modified live strain should be capable of limited replication in the vaccinated animal and of inducing a protective immune response which is protective against disease caused by virulent or wild-type CIAV.

The immunization method of the invention extends to the progeny of an immunized hen. The immune response in the hen produces antibodies in the hen that are passed to the chick through the egg. The antibodies are at sufficient titer to be protective against infection by CIAV of the progeny of immunized hens. Thus, the present CIAV vaccine prevents clinical disease in the progeny of immunized chickens by preventing CIAV infection in the chicks of immunized hens. The present vaccine can also be administered directly to chicks or embryos in ovo.

In the immunization method of the invention, the vaccine is administered to chickens prior to the onset of egg production. For example, a valid time range for most if not all types of chickens is from fertilization to about 12 weeks of age (and intervening days). The lower time is relevant based on the age-resistance phenomenon noted with CIAV. In chickens less than 4 weeks of age, non-Specific Pathogen Free (SPF) birds can be used. SPF chickens carry no maternal antibody or antibodies to the CIAV virus and therefore can be negatively impacted when exposed at a young age to the CIAV virus. However, non-SPF birds or commercially available broiler birds, which carry variable levels of CIAV maternal antibody, can benefit from exposure to the CIAV vaccine both prior to and after 4 weeks (Examples 11-13). Specifically, birds that are younger than 18 days of age can be vaccinated with CIAV vaccine. These chickens showed improved weight gain, shorter time to market, and a reduction in the number of pounds of poultry meat in demand at the processing plant. Specifically, the efficacy of the vaccine was measured by a statistically significant reduction in the rate of condemnation, a statistically significant increase flock viability, and a statistically significant decrease in pounds of meat in demand as measures of vaccine efficacy (Examples 11-13). The fact that the young vaccinated birds showed improvement in flock livability confirms the safety of using CIAV vaccine in young birds.

The CIAV vaccine can also be administered to chicken embryos in ovo. The in ovo administration of the vaccine involves the administration of the vaccine to eggs. There are numerous methods known in the art for administering a substance in ovo, which are discussed below. Eggs administered the vaccine of the present invention are fertile eggs which are preferably in the fourth quarter of incubation. Chicken eggs are treated on about the fifteenth to nineteenth day of incubation, and are most preferably treated on about the eighteenth or nineteenth day of incubation (the eighteenth or nineteenth day of embryonic development).

Non-SPF chickens can be vaccinated at any age since they are expected to have some resistance based on the presence of antibodies developed through maternal exposure. Similarly, for chicken types that develop resistance later, the vaccine can successfully be administered any time after resistance develops. Since resistance to CIAV disease can be routinely determined, for example, by using the methods shown in the Examples, this parameter is routinely adjustable, such that the invention is not limited to a particular lower age limit for immunization.

The upper time limit is relevant based on two general considerations: 1) the need to immunize sufficiently in advance of the onset of egg production to allow antibody titers to develop in the immunized hen; and 2) the need to immunize sufficiently in advance of the onset of egg production to allow clearance of the CIAV from the immunized hen. The age of onset of egg production varies among the different types of chickens. Thus, while 24 weeks is the approximate time of onset in the chickens tested, this parameter is not limited to that particular age, but is based on the routinely determinable age of onset for a given population of chickens.

In terms of the development of sufficient antibody titer, this is expected to vary within routinely determinable parameters from chicken to chicken. Thus, while 6 weeks prior to the onset of egg production has been determined to be sufficient in the strains tested, the contemplated time frame encompasses any time that can be determined to be sufficient for antibody production, including about 1, 2, 3, 4, 5, 6, 7, 8, 9, 10, 11, 12, 13, 14, 15, 16, 17, 18, 19, 20, 21, 23, 24 weeks (and intervening days) in advance of egg production. Methods of measuring antibody titer and determining sufficiency for protective immunization of progeny are routine and are provided in the Examples herein. h-i terms of the time needed to clear the virus prior to egg production, this is expected to vary within routinely determinable parameters from chicken to chicken. For the chickens exemplified herein, the it was determined that 12 weeks prior to egg productions is sufficient to clear the virus. Because this parameter is also routinely measured, the time frame contemplated encompasses any time sufficient to clear the virus, including about 1, 2, 3, 4, 5, 6, 7, 8, 9, 10, 11, 12, 13, 14, 15, 16, 17, 18, 19, 20, 21, 23, 24 weeks (and intervening days) in advance of egg production. Methods of measuring virus titer and determining clearance of the virus are routine and are provided in the Examples herein.

It should also be noted that the upper and lower time limits for administration of the vaccine are not necessarily based on the egg production status, antibody titer or virus titer of an individual chicken. Rather, it is the overall status of the group (e.g., population, strain, etc.) of chickens to be immunized that is relevant. Thus, if a sufficient percentage of individual chickens within a group are known or are expected (e.g., based on prior knowledge of the group) to be at the appropriate age for immunization, the immunization is considered successful.

The CIAV vaccine of the invention can be administered in combination with Marek's disease vaccine, infectious bursal disease vaccine, reovirus vaccine, Newcastle disease vaccine, infectious bronchitis disease vaccine, pneumovirus vaccine and avian influenza virus vaccine. Such vaccines are known in the art. The combination vaccination can be in the form of concurrent (or approximately concurrent) vaccination with separate vaccine preparations, or it can be in the form of a single formulation containing all of the desired vaccines.

The CIAV vaccine of the invention can be administered using any of the typical methods. For example, an advantageous method is to administer the vaccine in drinking water. The key features of the present water administered CIAV vaccine are 1) the CIAV is apathogenic for the host and is sufficiently invasive (at an acceptable input) to induce an adequate level of antibody; 2) the CIAV was demonstrated to spread; 3) the antibody induced will prevent the vertical transmission of a challenge virus; 4) the maternal antibody is efficiently transferred to the progeny and is protective; and 5) the antibody will endure for an extended period of time. The present data strongly support the premise that the CIAV possesses these key features.

Animals may be administered vaccines of the present invention by any suitable means. The vaccine may include carriers, thickeners, diluents, buffers, preservatives, surface active agents and the like in addition to the molecule of choice. The vaccine may also include one or more active ingredients such as antimicrobial agents, antiinflammatory agents, anesthetics, and the like.

When the animal to be treated is a bird, the bird may be a hatched bird, including a newly hatched (i.e., about the first three days after hatch), adolescent, and adult birds. Birds may be administered the vaccine in ovo, as described in U.S. Pat. No. 4,458,630 (the disclosure of this and all other patent references cited herein is to be incorporated herein by reference).

Eggs may be administered the vaccine of the invention by any means which transports the compound through the shell. The preferred method of administration is, however, by injection. The site of injection is preferably within either the region defined by the amnion, including the amniotic fluid and the embryo itself, in the yolk sac, or in the air cell. Most preferably, injection is made into the region defined by the amnion. By the beginning of the fourth quarter of incubation, the amnion is sufficiently enlarged that penetration thereof is assured nearly all of the time when the injection is made from the center of the large end of the egg along the longitudinal axis.

The mechanism of egg injection is not critical, but it is preferred that the method not unduly damage the tissues and organs of the embryo or the extraembryonic membranes surrounding it so that the treatment will not decrease hatch rate. A hypodermic syringe fitted with a needle of about 18 to 22 gauge is suitable for the purpose. To inject into the air cell, the needle need only be inserted into the egg by about two millimeters. A one inch needle, when fully inserted from the center of the large end of the egg, will penetrate the shell, the outer and inner shell membranes enclosing the air cell, and the amnion. Depending on the precise stage of development and position of the embryo, a needle of this length will terminate either in the fluid above the chick or in the chick itself. A pilot hole may be punched or drilled through the shell prior to insertion of the needle to prevent damaging or dulling of the needle. If desired, the egg can be sealed with a substantially bacteria- impermeable sealing material such as wax or the like to prevent subsequent entry of undesirable bacteria.

It is envisioned that a high speed automated egg injection system for avian embryos will be particularly suitable for practicing the present invention. Numerous such devices are available, exemplary being those disclosed in U.S. Pat. No. 4,681,063, U.S. Pat. Nos. 4,040,388, 4,469,047, and 4,593,646. These devices comprise an injection apparatus for delivering fluid substances into a plurality of eggs and suction apparatus which simultaneously engages and lifts a plurality of individual eggs from their upwardly facing portions and cooperates with the injection means for injecting the eggs while the eggs are engaged by the suction apparatus.

Alternatively, administration may be topically (including ophthalmically, vaginally, rectally, intranasally), orally, by inhalation, or parenterally, for example by intravenous drip, subcutaneous, intraperitoneal or intramuscular injection. The vaccine can also be administered subcutaneously, intracavity, or transdermally, or by aerosol spray (e.g., of any mucous membrane: nasal, pharyngeal, oral, ocular, intratracheal, cloacal, etc).

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional carriers, aqueous, powder or oily bases, thickeners and the like may be necessary or desirable.

Vaccines for oral administration include powders or granules, suspensions or solutions in water or non-aqueous media, capsules, sachets, or tablets. Thickeners, flavorings, diluents, emulsifiers, dispersing aids or binders may be desirable.

The invention provides a method of making a CIAV vaccine in an oncogenic cell line comprising subjecting the cell-cultured virus to more than one cycle of freezing and thawing, followed by maintaining the cells for about 3 days at about 37° C., whereby contaminating virus from the cell line is killed. There are numerous oncogenic cell lines that have growth characteristics and other characteristics that make them advantageous for growing CIAV. However, due to the existence in some of these cell lines of contaminating viruses (e.g., the tumor virus associate with the tumor from which the cell line was isolated), using them to produce a live CIAV vaccine has been problematic. The invention addresses this problem by providing methods of inactivating the contaminating virus without killing the CIAV. These methods are described in the Examples and elsewhere herein. Thus, the invention also provides a CIAV vaccine, comprising live CIAV passaged in an oncogenic cell line, wherein the vaccine does not cause Marek's Disease.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### Example 1

### Steps in Making the CIAV Vaccine in MSB-1 Cells

MSB-1 cells are maintained in vials frozen in liquid nitrogen until such time they are needed to expand into significant number for the propagation of the CIAV.

MSB-1 cells are planted as described in the scientific literature into various tissue culture vessels in RPMI-1640 media supplemented with fetal calf serum. Cells are incubated at about 41° C. These cells grow rapidly and can be frequently expanded to maintain actively growing cells.

The vaccine is produced by adding the CIAV virus to cells that have been expanded into new media such that the cell density is approximately 1 to 5×10⁵ cells/ml media, and the virus input is at least about 1×10⁵ TCID₅₀/ml media.

The virus-infected cells are incubated at about 41° C. for 4 to 7 days. Cells are microscopically examined for evidence of cell death as the determination of harvest time.

A step can be added to the virus harvest procedure to ensure inactivation of any residual Marek's disease virus that may be in the MSB-1 cells or that may be cell free. A proven effective procedure is the filtering of the cells and media through a Pall 4.5 to 5 micron cartridge to remove the MSB-1 cells followed by temperature treatment of the virus for about three days at about 37° C. to ensure inactivation of cell-free Marek's disease virus. Alternatively, the virus may be frozen and thawed three times to sufficiently rupture the MSB-1 cells to release and inactivate Marek's disease virus (an obligate intracellular pathogen). Then the virus fluid is subjected to a temperature treatment of about 37° C. for 3 days to ensure complete inactivation of any residual Marek's disease virus.

Since the CIAV is very stable the vaccine can be supplied in a frozen form or in liquid form kept at refrigerated temperature of 2-7° C., or the virus may be freeze-dried.

### Example 2

### PCR and Restriction Analysis

### Preparation of Intervet CIAV Vaccine Sample in MSB-1 Cells

Due to the incompatibility of the blue dye contained in the Intervet CIAV chicken embryo-adapted and attenuated vaccine sample (Intervet CIAV) and the PCR test, the sample was passed once in MSB-1 cells. MSB-1 cells were inoculated with 1:2 and 1:10 dilutions of virus, and cells were incubated for 96 hours prior to harvest. The culture media still appeared blue due to the dye in the vaccine sample so the cells were separated from the supernatant by centrifugation and the cells were washed twice with PBS. Both supernatant and cells were stored at −70° C.

### PCR

CIAV PCR following the protocol of the Center for Veterinary Biologics Laboratory (CVBL) in Ames, IA was conducted on the following samples:
1) CIAV, Del Ros strain
2) Intervet embryo-adapted commercial CIAV vaccine (Intervet CIAV), serial no. 2448003
3) MSB-1 cells of passage 1 (P1) of Intervet CIAV passaged at a 1:2 dilution
4) Supernatant of P1 passaged at a 1:2 dilution
5) MSB-1 cells of passage 1 (P1) of Intervet CIAV passaged at a 1:10 dilution
6) Supernatant P1 passaged a 1:10 dilution
7) MSB-1 cells only

The primers are: 5′ CTA/AGA/TCT/GCA/ACT/GCG/GA 3′ and 5′ CCT/TGG/AAG/CGG/ATA/GTC/AT 3′

### Restriction Enzyme Analysis.

Part of the CVBL protocol to further verify CAV, uses restriction enzyme analysis with HindIII, which states that the PCR product is cut one time. For restriction enzyme analysis, the PCR products were cut out of the agarose gel and the DNA was purified. Then the products from the cell samples were combined with the supernatant samples before cutting with HindIII. Results can be seen in Table 1.

TABLE 1
PCR amplification and restriction enzyme analysis.

| | PCR | |
|---|---|---|
| | positive/negative | HindIII |
| Sample | (bp) | fragments |
| CIAV, Del Ros strain | positive (419 bp) | 281 and 138 bp |
| Intervet CIAV | positive (419 bp) | 419 bp |
| 1:2 dilution of P1 - cells | positive (419 bp) | 419 bp |
| 1:2 dilution of P1 - supernatant | positive (419 bp) | |
| 1:10 dilution of P1 - cells | positive (419 bp) | 419 bp |
| 1:10 dilution of P1 - supernatant | positive (419 bp) | |
| MSB-1 cells only | Negative | N/A |

The primers used by CVBL were designed to the Cuxhaven-1 isolate which amplifies a 419 bp region starting at nucleotide 654 and ends at nucleotide 1072 of the genomic DNA-plus strand. This region overlaps 3 ORF's of which one encodes for VP-1, capsid protein. These primers amplified the sample. Surprisingly, the restriction enzyme that normally cuts the PCR product did not cut this sample. This means that the sample is probably CAV due to amplification by the primers, but it is different from the Del Ros (Delaware), Cl-1 (Maryland), Cuxhaven-1 (Germany), and the Gifu-1 isolate (Japan). The difference in the nucleotide sequence may be just one base change at the HindIII site such that the enzyme's recognition site has been altered. The difference may also be due to many base changes, but DNA sequencing of the PCR product would be needed to determine the similarity between the Del Ros strain and the sample.

### Example 3

### Results of CIAV-DR Bird Studies

### Pathogenicity Evaluation of the CIAV, Del-Ros Strain (CIAV-DR)

2-day-old, CAV-negative SPF chicks; 20 inoculates, 10 negative controls; 10^{6.9} TCID₅₀ of CIAV-DR in 0.2 ml; per os were used. The clinical and serological results can be seen in Table 2.

TABLE 2
Pathogenicity Evaluation

| | Week p.i/% | | | | | | | | | |
|---|---|---|---|---|---|---|---|---|---|---|
| | Weight | | | Day p.i. (dpi) | | | | | Day p.i./ | |
| | Reduction | | | Hemat. Val. | | | | | ELISA (total) | |
| Treat. | 1 | 2 | 3 | 14 | 21 | 28 | % Mort. | % Gross Les. | 28 | 35 |
| Negative | 0 | 0 | 0 | 39 | 35 | 36^{a} | 40 (NS^{b}) | 0 | 1/6 | 0/6 |
| Control | | | | | | | | | | |
| CAV | 0 | 9 | 0 | 32 | 31 | 34 | 0 | 30 | 19/20 | 20/20 |
| Del-Ros | | | | | | | | | | |

^{a}Mean hematocrit values
^{b}Non-specific

This study demonstrated that the Del-Ros strain is of low virulence because of the fact that it had little or no impact on growth rate, anemia, mortality and gross lesions when administered to the most susceptible age, CIAV-negative chickens by a natural route (i.e., oral). However, Del-Ros strain was sufficiently invasive to induce a good antibody response (i.e., 100% ELISA positive; VN titers ranging from 1:256-1:1024. The gross lesions observed were restricted to hemorrhages of muscles and pale bone marrow.

### Pathogenicity Evaluation of 3 Atrains of CIAV; Del-Ros, CA V-9 and Texas

2-day-old, CAV-negative SPF chicks; 10 chicks per virus strain, 5 negative controls; approx. 105.7 TCID50 of virus in 0.2 ml; IA was used. The clinical and serological results can be seen in Table 3.

TABLE 3
Pathogenicity Evaluation

| | | | | | | | | | Day p.i./ |
|---|---|---|---|---|---|---|---|---|---|
| | Week p.i/% Weight | | | Day p.i./ | | | | % | ELISA |
| | Reduction | | | Hemat. Val. | | | % | Gross | (total) |
| Treat. | 1 | 2 | 3 | 14 | 21 | 28 | Mort. | Les. | 28 |
| Control | 0 | 0 | 0 | 37 | 33 | 33* | 0 | 0 | 0/5 |
| Del-Ros | 0 | 9 | 0 | 32 | 31 | 34 | 0 | 30 | 9/10 |
| CAV-9 | 32 | 0 | 4 | 29 | 28 | 30 | 50 | 70 | 5/5 |
| Texas | 29 | 0 | 1 | 24 | 25 | 34 | 70 | 70 | 3/3 |

*Mean hematocrit values

This study demonstrated that the Texas strain of CIAV was sufficiently virulent to be used as a challenge virus when inoculated into 1- or 2-day-old susceptible chicks by a parenteral route (e.g., intra-abdominal). The gross lesions observed included; thymic atrophy, subcutaneous and intramuscular hemorrhaging, pale bone marrow, enlarged end congested liver lobes and gangrenous dermatitis.

### Example 4

### A Study Conducted with Chicken Infectious Anemia Virus, Del Ros Strain, by Serial Back Passaging in SPF Chickens to Demonstrate Virus does not Become Virulent

A host animal reversion to virulence study was conducted on the chicken infectious anemia virus, Del Ros strain (CIAV-DR) by serial backpassage in CIAV serologically negative SPF chickens.

The potential reversion to virulence of the CIAV-DR live vaccine by serial backpassage in the host animal was evaluated by daily observations for clinical signs, hematocrit value determinations and postmortem examinations for gross lesions characteristic of CIA.

Chickens used in the reversion to virulence study were CIAV-negative, SPF leghorn-type purchased from SPAFAS, Storrs CT. Three-week-old chickens were delivered banded for identification and at that time all were bled for CIAV serology to determine the CIAV serological status (ELISA; IDEXX CAV Kit) of the birds. At four weeks of age, eight to thirteen (backpassages 2-4) or twenty-four to twenty-eight (backpassages 1 and 5) chickens per virus backpassage were vaccinated with a 10 μl dose (10^{5.8} TCID₅₀, 1^{st} baclkpassage; a 20% suspension of a pooled tissue homogenate from the preceding backpassage given at a rate of 10 μl/bird, 2nd through 5th backpassage) via the wing web route. This series of five backpassages occurred over an eight-week period.

Liver, spleen and thymus were removed from eight euthanized chickens per backpassage at seven days post vaccination (DPV) to prepare a 20% suspension of a pooled tissue homogenate (Waring Blender) in RPMI 1640 medium containing antibiotics, but no serum and used as working stock in the inoculation of chickens for backpassage and virus isolation in MSB-1 cells according to the procedure of Yuasa et al. [Natl. Inst. Anim. Health Q (Tokyo) 23:75-77,1983].

All of the chickens of each backpassage were observed daily for clinical signs for seven (backpassages 2-4) or twenty-one DPV and the findings recorded. Blood was collected from all remaining chickens in backpassage one and five at fourteen and twenty-one DPV for hematocrit value determination. Chickens euthanized at seven and twenty-one DPV were examined for gross lesions characteristic of CIA. An analysis of phenotypic stability was conducted on the virus recovered form the fifth backpassage in chickens as compared by standard indirect fluorescent antibody assay (IFA).

The results obtained reveal that the CIAV-DR did not induce morbidity and mortality, aneirua and gross lesions characteristic of CIA when subjected to five serial backassages in chickens. Additionally, it was demonstrated that the CIAV remained phenotypically stable in the process.

Results of pre-trial blood samples for CIAV serological status, virus recovery from tissue homogenate extracts and post-mortem and hematocrit value findings at seven, fourteen and twenty-one DPV for the five backpassages are given in tables 4-8.

A sunmmary of the virus recovery, hematocrit value and post-mortem examination results are given in Table 9.

This reversion to virulence study conducted with a live CIAV-DR, administered by wing web to four week old chickens, demonstrated that the virus did not revert to virulence when subjected to five serial backpassages, based on clinical observations and postmortem examinations.

TABLE 4
ELISA, Virus Recovery, Hematocrit and Post-mortem
Results for the First Serial Backpassage.

| | | | Hematocrit | |
|---|---|---|---|---|
| Bird No. | Band No. | ELISA S/N | 14 d/21 d | CIAV SGL*** |
| 1 | 1 | 1.09* | 35/30** | None |
| 2 | 2 | 1.15 | 29/31 | None |
| 3 | 3 | 1.13 | 30/30 | None |
| 4 | 4 | 1.16 | 30/33 | None |
| 5 | 5 | 1.25 | NA/NA | None |
| 6 | 6 | 1.24 | 35/36 | None |
| 7 | 7 | 1.31 | NA/NA | None |
| 8 | 8 | 0.91 | NA/NA | None |
| 9 | 9 | 0.77 | 34/29 | None |
| 10 | 10 | 1.06 | 30/31 | None |
| 11 | 11 | 1.14 | 34/32 | None |
| 12 | 12 | 1.25 | 30/31 | None |
| 13 | 14 | 1.32 | NA/NA | None |
| 14 | 15 | 1.13 | 34/37 | None |
| 15 | 16 | 0.95 | 31/27 | None |
| 16 | 17 | 1.08 | NA/NA | None |
| 17 | 18 | 1.14 | 32/30 | None |
| 18 | 19 | 1.2 | 32/34 | None |
| 19 | 20 | 1.3 | 34/31 | None |
| 20 | 21 | 1.35 | NA/NA | None |
| 21 | 22 | 1.41 | NA/NA | None |
| 22 | 23 | 0.96 | NA/NA | None |
| 23 | 25 | 1.1 | 30/28 | None |
| 24 | 26 | 1.18 | 34/32 | None |
| 25 | 27 | 1.29 | 32/32 | None |
| 26 | 29 | 1.39 | 29/30 | None |
| 27 | 30 | 1.38 | 35/34 | None |
| 28 | 31 | 1.04 | 32/33 | None |

Virus Recovery from a Pooled Tissue Homogenate = Positive
*S/N Ratios > 0.6 = Negative (IDEXX Kit Interpretation)
**Hematocrit Value > 25 = Negative
***Specific Gross Lesions

TABLE 5
ELISA, Virus Recovery and Post-mortem Results
for the Second Serial Backpassage.

| Bird No. | Band No. | ELISA S/N | CIAV SGL** |
|---|---|---|---|
| 1 | 32 | 1.06* | None |
| 2 | 33 | 1.1 | None |
| 3 | 34 | 1.02 | None |
| 4 | 35 | 0.93 | None |
| 5 | 36 | 1.01 | None |
| 6 | 37 | 0.98 | None |
| 7 | 38 | 1.03 | None |
| 8 | 39 | 1 | None |
| 9 | 40 | 0.97 | None |
| 10 | 41 | 0.99 | None |
| 11 | 42 | 1 | None |
| 12 | 43 | 0.96 | None |
| 13 | 44 | 0.93 | None |

Virus Recovery from a Pooled Tissue Homogenate = Positive
*S/N Ratios > 0.6 = Negative (IDEXX Kit Interpretation)
***Specific Gross Lesions

TABLE 6
ELISA, Virus Recovery and Post-mortem Results for the Third
Serial Backpassage.

| Bird No. | Band No. | ELISA S/N | CIAV SGL** |
|---|---|---|---|
| 1 | 45 | 0.9* | None |
| 2 | 46 | 0.94 | None |
| 3 | 47 | 0.61 | None |
| 4 | 48 | 0.78 | None |
| 5 | 49 | 0.7 | None |
| 6 | 50 | 0.84 | None |
| 7 | 51 | 0.83 | None |
| 8 | 52 | 0.97 | None |
| 9 | 53 | 0.88 | None |
| 10 | 54 | 0.81 | None |
| 11 | 55 | 0.78 | None |
| 12 | 56 | 0.83 | None |
| 13 | 57 | 0.85 | None |

Virus Recovery from a Pooled Tissue Homogenate = Positive
*S/N Ratio > 0.6 = Negative (IDEXX Kit Interpretation)
**Specific Gross Lesions

TABLE 7
ELISA, Virus Recovery and Post-mortem Results the
Fourth Serial Backpassage.

| Bird No. | Band No. | ELISA S/N | CIAV SGL** |
|---|---|---|---|
| 1 | 59 | 0.93* | None |
| 2 | 60 | 0.9 | None |
| 3 | 61 | 0.86 | None |
| 4 | 62 | 0.9 | None |
| 5 | 63 | 0.88 | None |
| 6 | 64 | 0.87 | None |
| 7 | 67 | 0.83 | None |
| 8 | 70 | 0.95 | None |

Virus Recovery from a Pooled Tissue Homogenate = Positive
*S/N Ratio > 0.6 = Negative (IDEXX Kit Interpretation)
**Specific Gross Lesions

TABLE 8
Summary of Hematocrit, Virus Recovery and
Post-mortem Results of Chickens.

| Back | | Virus | |
|---|---|---|---|
| Passage | Hematocrit | Recovery | Post-Mortem |
| 1 | 0/20* | 1/1** | 0/28 |
| 2 | — | 1/1 | 0/13 |
| 3 | — | 1/1 | 0/13 |
| 4 | — | 1/1 | 0/8 |
| 5 | 0/16 | 1/1 | 0/24 |

*Number Positive/Number in Group
**Virus Recovery for a Pooled Tissue Homogenate

TABLE 9
ELISA, Virus Recovery, Hematocrit and Post-mortem Results for the
Fifth Serial Backpassage.

| | | | Hematocrit | |
|---|---|---|---|---|
| Bird No. | Band No. | ELISA S/N | 14 d/21 d | CIAV SGL*** |
| 1 | 2 | 0.81* | NA/NA | None |
| 2 | 3 | 0.61 | 32/34** | None |
| 3 | 4 | 0.72 | 36/31 | None |
| 4 | 5 | 0.79 | 33/32 | None |
| 5 | 6 | 0.87 | 32/35 | None |
| 6 | 7 | 1.09 | NA/NA | None |
| 7 | 9 | 0.7 | 34/35 | None |
| 8 | 10 | 0.79 | NA/NA | None |
| 9 | 11 | 0.9 | NA/NA | None |
| 10 | 13 | 0.93 | 31/33 | None |
| 11 | 14 | 1.03 | NA/NA | None |
| 12 | 15 | 0.97 | 32/35 | None |
| 13 | 18 | 0.8 | 26/30 | None |
| 14 | 19 | 0.84 | 35/33 | None |
| 15 | 20 | 0.92 | 33/33 | None |
| 16 | 21 | 0.91 | 26/32 | None |
| 17 | 23 | 1.05 | 29/35 | None |
| 18 | 24 | 0.61 | NA/NA | None |
| 19 | 25 | 0.89 | 28/35 | None |
| 20 | 26 | 0.92 | 30/30 | None |
| 21 | 28 | 0.97 | NA/NA | None |
| 22 | 29 | 0.96 | 33/35 | None |
| 23 | 30 | 0.99 | 32/35 | None |
| 24 | 31 | 0.95 | NA/NA | None |

Virus Recovery from a Pooled Tissue Homogenate = Positive
*S/N Ratio > 0.6 = Negative (IDEXX Kit Interpretation)
**Hematocrit Value > 25 = Negative
***Specific Gross Lesions

### Example 5

### Results of a Shed/Spread and Vertical Transmission Study Conducted in SPF Chickens Following Wing Web Administration

A host animal shed/spread and vertical transmission study was conducted in chicken infectious anemia virus (CIAV)-negative, SPF chickens on a chicken infectious anemia virus, Del Ros strain, (CIAV-DR) administered by the wing web route. To assess shed and spread of CIAV live vaccine to contact controls, cloacal swabs were collected from vaccinated and contact control chickens for a 4 week post vaccination (p.v.) period and assayed for virus isolation in MSB-1 cells. To evaluate vertical transmission (i.e., p.v.) of CIAV live vaccine, pools of livers of 19-day-old embryos derived from eggs laid by vaccinated hens were assayed for virus by isolation in MSB-1 cells and by PCR detection.

The methods used to determine the shed/spread and vertical transmission of a new CIA master seed virus were conducted in CIAV-negative, SPF chickens vaccinated at 12 weeks of age. The possible shed and spread of wing web administered CIAV vaccine (live virus) was evaluated by collecting cloacal swabs from vaccinated and contact control chickens for a 4 week p.v. period followed by virus isolation attempts in MSB-1 cells. The possibility of vertical transmission of live CIAV vaccine was examined by assaying pools of livers of 19-day-old embryos derived from all of the fertile eggs laid by all of the vaccinated hens for virus by isolation in MSB-1 cells and by PCR detection. Livers of embryos from 3 settings of eggs from negative control hens were evaluated in the same manner.

Chickens used in the shed/spread and vertical transmission study were CIAV-negative, SPF leghorn-type (SPF flock L103) purchased from SPAFAS. Birds were banded for identification. Ten randomly selected chickens at 12 weeks of age were bled for CIAV serology to confirm the negative status (ELISA; IDEXX CAV Kit) of the birds. On the same day, thirty-seven chickens (30 females and 7 males) were vaccinated with a 10 μl dose (10^{4.3} TCID₅₀) of the live CIAV vaccine by the wing web route. Fifteen females (same source and hatch) were intermixed with the vaccinates as contact controls. Negative control chickens from the same source and hatch were maintained. Chickens of both groups were observed daily for morbidity and mortality and findings recorded for the duration of the study period.

Cloacal swab collections from fifteen randomly selected vaccinated chickens and the fifteen contact controls were made at 3-7 day intervals for a 4 week p.v. period. Cloacal swabs were pooled for virus reisolation by combining 3 groups of 5 swabs per treatment per sampling time. Virus recovery attempts were made in MSB-1 cells according to the procedure of Yuasa et al. [Natl. Inst. Anim. Health Q (Tokyo) 23:75-77, 1983].

Livers were aseptically collected from live and dead embryos (derived from fertile eggs laid by vaccinated and negative control hens for a 3 week p.v. period) at 19 days of incubation and packaged/ stored (−20° C.) in pools of 3-6 livers for future processing. Twenty percent (w/v) liver (pools) suspensions were prepared in RPMI 1640 mediun plus 5% FBS for virus reisolation in MSB-1 cells according to the procedure of Yuasa et al. [Natl. Inst. Anim. Health Q (Tokyo) 23:75-77, 1983]. Prior to initiating a CIAV isolation procedure on test hens, an assessment of the sensitivity of the CIAV isolation method outlined in the “shed/spread and vertical transmission protocol” was conducted. Briefly, this procedure entailed harvesting livers from CIAV-antibody free SPF embryos at 19 days of incubation and preparing four pools of five livers each. One liver pool was maintained as a negative control; second, third and fourth pools were inoculated with 10, 100 and 1000 TCID₅₀ of CIAV per gram of tissue, respectively.

In addition to virus reisolation assays conducted, attempts to detect CIAV by PCR according to the procedure of Taylor and Ryncarz (Center for Veterinary Biologics Laboratory, NVSL, VS, APHIS, USDA, Ames, IA) were undertaken.

The results revealed that 10^{4.3} TCID₅₀ of the CIAV-DR administered to breeders at 12 weeks of age via the wing web is shed for as much as 21 days and that it will spread to contact controls. However, the virus was not vertically transmitted by breeders to their progeny as demonstrated by virus reisolation and PCR assays. The breeders did not exhibit any adverse clinical effects from the vaccine administration.

Results of ELISA on pre-trial blood samples confirmed that the chickens used in this study were CIAV-antibody negative (table 10).

Results of virus reisolation attempts on cloacal swab pools of vaccinates and contact controls are recorded in table 2. These data show that CIAV was being shed by vaccinates as soon as 3 days p.v. and this shed continued through 21 days p.v., but not 28 days p.v. Additionally, the data show that the shed CIAV readily spread to the contact controls who also shed the virus for similar period of time.

A summary of the virus reisolation and PCR detection attempts on embryo liver suspensions derived from the fertile eggs of vaccinates and negative controls are given in table 3. These data reveal that CIAV could not be isolated from embryo liver suspensions of negative control and vaccinates by passage in MSB-1 cells or be detected by PCR. The results of a CIAV isolation sensitivity assessment in MSB-1 cells demonstrated that varying levels of CIAV (i.e., 10-1000 TCID₅₀/gram of tissue) was detected by this method following several cell culture passages (table 4). There was complete correlation in the results obtained using these two methods on test samples.

This shed/spread and vertical transmission study was based on an effort to isolate and/or detect live CIAV in cloacal swabs and fertile eggs (i.e., embryo liver suspensions) collected from wing web vaccinated (10^{4.3} TCID₅₀/dose) and negative control hens. The results demonstrated that the virus was shed and spread for a limited period of time (21 dpv) but that this virus was not transmitted vertically when administered at 12 weeks of age.

TABLE 10
Pre-Trial Blood Sample ELISA Results.

| Bird No. | Band No. | S/N Ratio | CIAV Serol. Status |
|---|---|---|---|
| 1 | 554 | 0.91 | Neg^{a} |
| 2 | 557 | 0.93 | Neg. |
| 3 | 565 | 0.92 | Neg. |
| 4 | 566 | 0.96 | Neg. |
| 5 | 574 | 0.96 | Neg. |
| 6 | 579 | 1 | Neg. |
| 7 | 584 | 1 | Neg. |
| 8 | 585 | 1 | Neg. |
| 9 | 731 | 0.99 | Neg. |
| 10 | 740 | 0.99 | Neg. |

^{a}Negative = S/N Ratio > 0.6 (IDEXX Kit Interpretation)

TABLE 11
Shed/Spread: Summary of Virus Reisolation from Cloacal Swab
Pools of Vaccinated and Contact Control Chickens.

| | | | | Contact | | |
|---|---|---|---|---|---|---|
| | Vaccinate | | | Control Cloacal | | |
| Cloacal | Cloacal Swab Pools | | | Swab Pools | | |
| Swab (dpv)^{a} | 1 | 2 | 3 | 1 | 2 | 3 |
| 3 | N^{b} | P^{c} | N | N | N | P |
| 7 | P | P | P | P | P | P |
| 12 | N | P | N | N | P | N |
| 16 | P | P | N | N | N | P |
| 21 | P | P | P | P | N | N |
| 28 | N | N | N | N | N | N |

^{a}Cloacal Swab Collection (Days Post Vaccination).
^{b}Negative
^{c}Positive = Characteristic CIAV CPE Observed

TABLE 12
Vertical Transmission: Summary of Virus Reisolation and PCR
Detection Assays on Embryo Liver Suspensions Derived from
the Fertile Eggs of Vaccinates and Negative Controls.

| Treatment | Virus Reisolation | PCR Detection |
|---|---|---|
| 1^{a} | 0/12^{b} | 0/12 |
| 2a^{c} | 0/17 | 0/17 |
| 2b | 0/15 | 0/15 |
| 2c | 0/19 | 0/19 |
| 2d | 0/18 | 0/18 |
| Pos. Con.^{d} | 6/6 | 5/6 |
| Neg. Con.^{e} | 0/6 | 0/6 |

^{a}SPAFAS Negative Controls
^{b}Number Positive/Total Tested
^{c}Vaccinates - four groups (2a-2d)
^{d}Positive Controls (MSB-1 Propagated Del-Ros and Texas Strains of CIAV)
^{e}Negative Controls (MSB-1 cells and/or Reagent Mix)

TABLE 13
Results of a CIAV Isolation Sensitivity Assessment.

MSB-1 Passages

| Treatment | 1 | 2 | 3 | 4 | 5 | 6 |
|---|---|---|---|---|---|---|
| 10 TC1D₅₀^{a} | 0/5^{b} | 0/5 | 0/5 | 0/5 | 0/5 | 5/5 |
| 100 TC1D₅₀ | 0/5 | 0/5 | 0/5 | 0/5 | 0/5 | 5/5 |
| 1000 TC1D₅₀ | 0/5 | 0/5 | 0/5 | 0/5 | 3/5 | 5/5 |
| Uninf. Cont.^{c} | 0/5 | 0/5 | 0/5 | 0/5 | 0/5 | 0/5 |

^{a}TCID₅₀/Gram of Tissue
^{b}Number Positive (Characteristic CIAV CPE Observed)/Total
^{c}Uninfected Controls

### Example 6

### Efficacy Study Conducted in Progeny of SPF Chickens 34 and 39 Weeks Following Wing Web Administration of a Live Chicken Anemia Virus Vaccine

A study to evaluate vaccine efficacy and duration of immunity (DOI) at 34 and 49 weeks post wing web vaccination was conducted by challenging day-old progeny of hens vaccinated with a chicken infectious anemia virus, Del Ros strain, (CIAV-DR). The study assessed CIAV maternal antibody protection (passive immunity) provided to chicks against a challenge with virulent CIAV.

Efficacy and duration of immunity of the were conducted in the progeny of CIAV-negative, SPF chickens vaccinated at 9 weeks of age with CIAV vaccine administered via the wing web route. Duration of immunity was evaluated by challenging progeny, hatched from fertile eggs laid by hens at 34 and 49 weeks post vaccination, followed by observations for clinical signs, hematocrit value determinations and post-mortem examinations for gross lesions characteristic of CIA.

Chickens used in this study were CIAV-negative, SPF leghorn-type purchased from SPAFAS. Birds were wing-banded for identification. Ten randomly selected chickens at 9-weeks-of-age were bled for CIAV serology to confirm the negative serological status (ELISA, IDEXX CAV Kit) of the birds. On the same day, 70 chickens (60 females and 10 males) were vaccinated with a 10 μl dose (10^{4.2} TCID₅₀) of the live CIAV vaccine by the wing web route. Negative control chickens from the same source and hatch were maintained. The dose was determined as the average of 5 replicate titers conducted immediately after vaccination. Chickens of both groups were observed daily for morbidity and mortality and the findings recorded for the duration of the study period.

A one-week collection of eggs from 52 vaccinated hens (43-weeks-of-age) were used to evaluate progeny of breeders at 34 weeks post CIAV vaccination (DOI Test 2). A second one-week collection of eggs from 48 vaccinated hens (58 weeks of age to assess progeny of breeders at 49 weeks post CIAV vaccination (DOI Test 3).

Forty-day-old chicks, each from CIAV vaccinated and non-vaccinated breeders, were challenged with liver homogenate extract derived from chicks inoculated with a Texas field isolate of CIAV. Each chick was inoculated intra-abdominally with approximately 10^{2.6} CID₅₀ per 0.2 ml. Negative control groups consisted of 25 chicks.

Chicks of all treatment groups were maintained in separate filtered-air, negative-pressure isolators and observed daily for depression, ruffled feathers and mortality. Blood samples were collected from all of the chicks at 14 and 21-22 days post challenge for hematocrit value determinations as a measure of anemia. The procedure used for determining hematocrit values was that of Rosenberger and Cloud (Avian Dis. 33:753-759, 1989). Additionally, chicks of all treatment groups were examined for gross lesions characteristic of CIA (i.e., pale bone marrow, swelling and discoloration of the liver and spleen and hemorrhagic lesions in the skin and muscles) at 21-22 days post challenge. Treatment comparisons were based on the number of individuals within a treatment (per total examined) exhibiting specific gross lesions of CIA.

The results of the two DOI challenge tests, reported herein, demonstrated that 1I042 TCID₅₀ of virus administered to breeders at 9 weeks of age via the wing web protected progeny against morbidity and mortality, anemia and gross lesions characteristic of CIA through 49 weeks post vaccination as determined by statistical evaluation.

Pre-study blood sample ELISA results were found to confirm the CIAV-negative status of the semi-mature chickens acquired from SPAFAS for use in this study and are presented in table 14.

Results of hematocrit value determinations, clinical-sign findings and post-mortem examinations of CIAV challenged and non-challenged day-old chicks are recorded in tables 15, 16 and 17 (DOI Test 2) and 20, 21, and 22 (DOI Test 3); tables 18 and 23, respectively, summarize this information. Chicks with gross lesion scores ≧1, for any one of the tissues examined (i.e., liver, muscle, bone marrow and thymus), were recorded as CIA positive (tables 18 and 23). The death of chicks (table 15; derived from CIAV vaccinated breeders) numbered 3, 8, 22, 26, 27 and 40 in DOI test 2 resulted from suffocation in an isolator glove. Statistical evaluations (Fisher's Exact Probability Test; tables 19 and 24) of hematocrit values and clinical signs of Test 2 and 3 chicks revealed that progeny of CIAV vaccinated versus non vaccinated breeders were protected against anemia and mortality at a statistically significant level (p <0.001) when challenged with a virulent field isolate of CIAV. A statistically significant difference (p=0.027) in morbidity was demonstrated among challenged progeny in DOI Test 3. Statistical assessment (Mann-Whitney Test; tables 20 and 25) of gross lesion scores revealed similar findings as those reported above; i.e., a statistically significant difference and in the bone marrow (p <0.001 and p=0.021, respectively) and thymus (p <0.001) gross lesion scores of progeny derived from vaccinated versus non-vaccinated breeders. No significant differences were demonstrated marrow (p <0.001 and p=0.021, respectively) and thymus (p <0.001) gross lesion scores of progeny derived from vaccinated versus non-vaccinated breeders. No significant differences were demonstrated for liver and muscle lesions among challenged progeny.

This assessment of vaccine efficacy and immunity duration was based on a day-old, intra abdominal challenge of progeny derived from breeders vaccinated at 9 weeks of age with live CIAV-DR vaccine administered by the wing web route. The results revealed that the CIAV vaccine induced maternal antibodies which protected chicks at a statistically significant difference of p <0.05, against a virulent challenge with a field strain of CIAV, based on evidence of anemia at 14 and 21 days post challenge, clinical signs and gross lesions of the bone marrow and thymus when compared to challenge control chicks.

TABLE 14
Pre-Trial Blood Sample ELISA Results of 9 Week Old Chickens
Prior to Vaccination with Wing Web Administered CIAV to
Confirm Negative Serological Status.

| Bird No. | Band No. | S/N Ratio | CIAV Serol. Status^{a} |
|---|---|---|---|
| 1 | 602 | 0.88 | Neg^{b} |
| 2 | 608 | 0.84 | Neg |
| 3 | 616 | 0.9 | Neg |
| 4 | 620 | 0.81 | Neg |
| 5 | 621 | 0.78 | Neg |
| 6 | 627 | 0.85 | Neg |
| 7 | 631 | 0.87 | Neg |
| 8 | 634 | 0.82 | Neg |
| 9 | 644 | 0.85 | Neg |
| 10 | 661 | 0.7 | Neg |

^{a}CIAV Serological Status
^{b}Negative = S/N Ratio > 0.6 (IDEXX Kit Interpretation)

TABLE 15
Test 2 Hematocrit Values, Clinical Signs and CIA Gross Lesion Scores of
Chicks Challenged at 34 Weeks Following Wing Web Administered CIA Vaccine.

| | Hematocrit Values | | Clin. Signs^{a} | Gross Lesion Scores | | | |
|---|---|---|---|---|---|---|---|
| Bird No. | 14 Day pc^{b} | 21 Day pc | Mor./Mort.^{c} | Liver | BM^{d} | Thymus | Muscle |
| 1 | 28 | 35 | N/N | O^{e} | 0 | 0 | 0 |
| 2 | 33 | 32 | N/N | 0 | 0 | 0 | 0 |
| 3 | 35 | ND^{f} | N/NCAM^{g} | 0 | 0 | 0 | 0 |
| 4 | 32 | 39 | N/N | 0 | 0 | 0 | 0 |
| 5 | 32 | 34 | N/N | 0 | 0 | 0 | 0 |
| 6 | 26 | 27 | N/N | 0 | 0 | 0 | 0 |
| 7 | 28 | 32 | N/N | 0 | 0 | 0 | 0 |
| 8 | 32 | ND | N/NCAM | 0 | 0 | 0 | 0 |
| 9 | 32 | 33 | N/N | 0 | 0 | 0 | 0 |
| 10 | 32 | 24^{h} | N/N | 0 | 2 | 1 | 1 |
| 11 | 26 | 12 | P/Nⁱ | 0 | 2 | 2 | 0 |
| 12 | 33 | 26 | N/N | 0 | 2 | 2 | 0 |
| 13 | 27 | 31 | N/N | 0 | 0 | 0 | 0 |
| 14 | 32 | 35 | N/N | 0 | 0 | 0 | 0 |
| 15 | 33 | 32 | N/N | 0 | 0 | 0 | 0 |
| 16 | 60 | 39 | N/N | 0 | 0 | 0 | 0 |
| 17 | 58 | 37 | N/N | 0 | 0 | 0 | 0 |
| 18 | 30 | 24 | N/N | 0 | 1 | 2 | 0 |
| 19 | 33 | 34 | N/N | 0 | 0 | 0 | 0 |
| 20 | 21 | 17 | N/N0 | 0 | 3 | 2 | 0 |
| 21 | 58 | 35 | N/N | 0 | 0 | 0 | 0 |
| 22 | 32 | ND | N/NCAM | 0 | 0 | 0 | 0 |
| 23 | 33 | 37 | N/N | 0 | 2 | 1 | 0 |
| 24 | 34 | 36 | N/N | 0 | 0 | 0 | 0 |
| 25 | 29 | 33 | N/N | 0 | 0 | 0 | 0 |
| 26 | 30 | ND^{f} | N/NCAM^{g} | O^{e} | 0 | 0 | 0 |
| 27 | 34 | ND | N/NCAM | 0 | 0 | 0 | 0 |
| 28 | 35 | 32 | N/N | 0 | 0 | 0 | 0 |
| 29 | 35 | 27 | N/N | 0 | 0 | 0 | 0 |
| 30 | 28 | 23^{h} | N/N | 0 | 1 | 2 | 0 |
| 31 | 30 | 31 | N/N | 0 | 0 | 0 | 0 |
| 32 | ND | ND | N/Pⁱ | 0 | 0 | 0 | 0 |
| 33 | 33 | 35 | N/N | 0 | 0 | 0 | 0 |
| 34 | 34 | 41 | N/N | 0 | 0 | 0 | 0 |
| 35 | 27 | 36 | N/N | 0 | 0 | 0 | 0 |
| 36 | 32 | 34 | N/N | 0 | 0 | 0 | 0 |
| 37 | 30 | 21 | N/N | 0 | 0 | 0 | 0 |
| 38 | 33 | 36 | N/N | 0 | 0 | 0 | 0 |
| 39 | 31 | 34 | N/N | 0 | 0 | 0 | 0 |
| 40 | 30 | ND | N/NCAM | 0 | 0 | 0 | 0 |
| Pos./Tot.^{j} | 1/39 | 6/33 | 1/40/1/34 | 0/40 | 7/40 | 7/40 | 1/40 |

^{a}Clinical Signs
^{b}Post Challenge
^{c}Morbidity (Depression and/or Ruffled Feathers)/Mortality
^{d}Bone Marrow
^{e}0 = Normal; 1 = Slight; 2 = Moderate; 3 = Severe Not Done
^{f}Not None
^{g}Negative/Non-CIAV Associated Mortality
^{h}Hematocrit Values of ≦25 = Anemia
ⁱNegative/Positive (CIAV Associated Mortality)
^{j}Number Positive/Total

TABLE 16
Test 2 Hematocrit Values, Clinical Signs and CIA Gross Lesion Scores of
Challenged Chicks from Non-Vaccinated Breeders.

| | Hematocrit Values | | Clin. Signs^{a} | Gross Lesion Scores | | | |
|---|---|---|---|---|---|---|---|
| Bird No. | 14 Day pc^{b} | 21 Day pc | Mor./Mort.^{c} | Liver | BM^{d} | Thymus | Muscle |
| 1 | 23^{e} | ND^{f} | N/P^{g} | 0^{h} | 2 | 3 | 1 |
| 2 | 18 | ND | N/P | 0 | 2 | 2 | 0 |
| 3 | 29 | 22 | N/N | 0 | 0 | 0 | 0 |
| 4 | 26 | 20 | P/N | 0 | 0 | 3 | 0 |
| 5 | 20 | ND | N/P | 0 | 3 | 2 | 1 |
| 6 | 28 | 26 | N/N | 0 | 0- | 0 | 0 |
| 7 | 21 | 57 | N/N | 0 | 0 | 3 | 0 |
| 8 | 20 | ND | N/P | 0 | 3 | 3 | 0 |
| 9 | 21 | 21 | N/N | 0 | 2 | 2 | 0 |
| 10 | 18 | ND | N/P | 0 | 3 | 3 | 2 |
| 11 | 32 | 24 | N/N | 0 | 2 | 1 | 0 |
| 12 | 21 | ND | N/P | 0 | 3 | 3 | 1 |
| 13 | 26 | 24 | N/N | 0 | 0 | 0 | 0 |
| 14 | 25 | 19 | N/N | 0 | 0 | 1 | 0 |
| 15 | 25 | 45 | N/N | 0 | 2 | 1 | 0 |
| 16 | 28 | 30 | N/N | 0 | 2 | 3 | 0 |
| 17 | 27 | 10 | P/N | 0 | 3 | 3 | 0 |
| 18 | 16 | ND | P/P | 0 | 2 | 2 | 0 |
| 19 | 22 | 25 | N/N | 0 | 0 | 0 | 0 |
| 20 | 18 | 24 | N/N | 0 | 0 | 2 | 2 |
| 21 | 20 | ND | N/P | 0 | 3 | 2 | 1 |
| 22 | 28 | 20 | N/N | 0 | 1 | 3 | 0 |
| 23 | 26 | 15 | P/N | 0 | 2 | 1 | 0 |
| 24 | 22 | 28 | N/N | 0 | 0 | 0 | 0 |
| 25 | 17 | ND | P/P | 2 | 3 | 3 | 2 |
| 26 | 24^{e} | 30 | N/N | O^{h} | 0 | 2 | 0 |
| 27 | 40 | 56 | N/N | 0 | 2 | 2 | 0 |
| 28 | 30 | 15 | N/N | 0 | 2 | 2 | 0 |
| 29 | 29 | 29 | N/N | 0 | 0 | 0 | 0 |
| 30 | 31 | 27 | N/N | 0 | 1 | 2 | 0 |
| 31 | 25 | 32 | N/N | 0 | 1 | 2 | 0 |
| 32 | 25 | 13 | P/N | 0 | 3 | 3 | 0 |
| 33 | 21 | 27 | N/N | 0 | 0 | 0 | 0 |
| 34 | 28 | 21 | N/N | 0 | 2 | 2 | 0 |
| 35 | 30 | 28 | N/N | 0 | 0 | 0 | 0 |
| 36 | 30 | ND^{f} | N/P^{g} | 0 | 3 | 3 | 1 |
| 37 | 28 | 23 | N/N | 0 | 0 | 0 | 0 |
| 38 | 70 | 13 | N/N | 0 | 2 | 1 | 0 |
| 39 | 23 | 25 | N/N | 0 | 0 | 1 | 0 |
| 40 | 25 | 27 | N/N | 0 | 0 | 0 | 0 |
| Pos./Tot.ⁱ | 22/40 | 17/30 | 6/40/10/40 | 1/40 | 24/40 | 30/40 | 8/40 |

^{a}Clinical Signs
^{b}Post Challenge
^{c}Morbidity (Depression and/or Ruffled Feathers)/Mortality
^{d}Bone Marrow
^{e}Hematocrit Values ≦25 = Anemia
^{f}Not None
^{g}Negative/Positive (CIAV Associated Mortality)
^{h}0 = Normal; 1 = Slight; 2 = Moderate; 3 = Severe
ⁱNumber Positive/Total

TABLE 17
Test 2 Hematocrit Values, Clinical Signs and CIA Gross Lesion Scores of
Chicks from Non-Vaccinated Breeders; Not Challenged.

| | Hematocrit Values | | Clin. Signs^{a} | Gross Lesion Scores | | | |
|---|---|---|---|---|---|---|---|
| Bird No. | 14 Day pc^{b} | 21 Day pc | Mor./Mort.^{c} | Liver | BM^{d} | Thymus | Muscle |
| 1 | 37 | 38 | N/N^{e} | 0^{f} | 0 | 0 | 0 |
| 2 | 38 | 35 | N/N | 0 | 0 | 0 | 0 |
| 3 | 35 | 30 | N/N | 0 | 0 | 0 | 0 |
| 4 | 40 | 35 | N/N | 0 | 0 | 0 | 0 |
| 5 | 36 | 37 | N/N | 0 | 0 | 0 | 0 |
| 6 | 38 | 36 | N/N | 0 | 0 | 0 | 0 |
| 7 | 35 | 36 | N/N | 0 | 0 | 0 | 0 |
| 8 | 28 | 38 | N/N | 0 | 0 | 0 | 0 |
| 9 | NS^{g} | 35 | N/N | 0 | 0 | 0 | 0 |
| 10 | 31 | NS | N/N | 0 | 0 | 0 | 0 |
| 11 | 36 | 36 | N/N | 0 | 0 | 0 | 0 |
| 12 | 37 | 35 | N/N | 0 | 0 | 0 | 0 |
| 13 | 36 | 33 | N/N | 0 | 0 | 0 | 0 |
| 14 | 31 | 42 | N/N | 0 | 0 | 0 | 0 |
| 15 | 39 | 40 | N/N | 0 | 0 | 0 | 0 |
| 16 | 35 | 37 | N/N | 0 | 0 | 0 | 0 |
| 17 | 40 | 36 | N/N | 0 | 0 | 0 | 0 |
| 18 | 35 | 33 | N/N | 0 | 0 | 0 | 0 |
| 19 | 32 | 35 | N/N | 0 | 0 | 0 | 0 |
| 20 | 33 | 35 | N/N | 0 | 0 | 0 | 0 |
| 21 | 30 | 45 | N/N | 0 | 0 | 0 | 0 |
| 22 | 39 | 39 | N/N | 0 | 0 | 0 | 0 |
| 23 | 34 | 40 | N/N | 0 | 0 | 0 | 0 |
| 24 | 33 | 38 | N/N | 0 | 0 | 0 | 0 |
| 25 | 35 | 27 | N/N | 0 | 0 | 0 | 0 |
| Pos./Tot.^{h} | 0/24 | 0/24 | 0/25/0/25 | 0/25 | 0/25 | 0/25 | 0/25 |

^{a}Clinical Signs
^{b}Post Challenge
^{c}Morbidity (Depression and/or Ruffled Feathers)/Mortality
^{d}Bone Marrow
^{e}Negative/Negative
^{f}0 = Normal; 1 = Slight; 2 = Moderate; 3 = Severe
^{g}No Sample
^{h}Number Positive/Total

TABLE 18
Summary of Test 2 Hematocrit, Morbidity, Mortality and CIA Gross
Lesion Scores of Challenged and Non-Challenged Chicks.

| Test Group | Hematocrit | Morbidity | Mortality | PM^{a} |
|---|---|---|---|---|
| CIAV | 6/39 (15%)^{c} | 1/40 (3%) | 1/34 (3%) | 7/40 (18%)^{d} |
| Vaccinated^{b} | | | | |
| Non- | 33/40 (83%) | 6/40 (15%) | 10/40 (25%) | 30/40 (75%) |
| Vaccinated^{b} | | | | |
| Negative | 0/25 | 0/25 | 0/25 | 0/25 |
| Control | | | | |

^{a}Post-Mortem CIA Gross Lesion Scores
^{b}Challenge Group
^{c}Number Chicks Positive/Total
^{d}Positive Chicks = Gross Lesion Scores ≧1

TABLE 19
Statistical Evaluation of Test 2 Hematocrit Values and CIA Clinical
Signs of Challenged Chicks using Fisher's Exact Probability Test.

| | Hematocrit Values | | | | |
|---|---|---|---|---|---|
| Test | 14 | | Clinical Signs | | |
| Group | Day pc^{a} | 21 Day pc | Morbidity | Mortality | Combined |
| CIAV | 1/39 | 6/33 | 1/40 | 1/34 | 6/40^{b} |
| Vaccinated | | | | | |
| Non- | 22/40 | 17/30 | 6/40 | 10/40 | 34/40 |
| Vaccinated | | | | | |
| p value | <0.001 | 0.002 | 0.054 | 0.007 | <0.001 |

^{a}Post Challenge
^{b}Combined Hematocrit Values and Clinical Signs

TABLE 20
Statistical Evaluation of Test 2 CIA Gross Lesion Scores of Challenged
Chicks from Vaccinated and Non-Vaccinated Breeders using the
Mann-Whitney Test

| | Gross Lesion Scores^{a} | | | | |
|---|---|---|---|---|---|
| | Liver | BM^{b} | Thymus | Muscle | Combined^{c} |
| p value | 0.847 | <0.001 | <0.001 | 0.173 | <0.001 |

^{a}Raw Data Found in Tables 2 and 3
^{b}Bone Marrow
^{c}Combined Gross Lesion Scores

TABLE 21
Test 3 Hematocrit Values, Clinical Signs and CIA Gross Lesion Scores of
Chicks Challenged at 49 Weeks Following Wing Web Administered CIA Vaccine.

| | Hematocrit Values | | | | | | |
|---|---|---|---|---|---|---|---|
| | Scores | | Clin. Signs^{a} | Gross Lesion | | | |
| Bird No. | 14 Day pc^{b} | 21 Day pc | Mor./Mort.^{c} | Liver | BM^{d} | Thymus | Muscle |
| 1 | 31 | 45 | N/N^{e} | O^{f} | 0 | 0 | 0 |
| 2 | 30 | 32 | N/N | 0 | 0 | 0 | 0 |
| 3 | 34 | 34 | N/N | 0 | 0 | 0 | 0 |
| 4 | 28 | 28 | N/N | 0 | 0 | 0 | 0 |
| 5 | 33 | 23g | N/N | 0 | 0 | 0 | 0 |
| 6 | 32 | 30 | N/N | 0 | 0 | 0 | 0 |
| 7 | 24 | 36 | N/N | 0 | 0 | 0 | 0 |
| 8 | 49 | 32 | N/N | 0 | 0 | 0 | 0 |
| 9 | 35 | 30 | N/N | 0 | 0 | 0 | 0 |
| 10 | 31 | 31 | N/N | 0 | 0 | 0 | 0 |
| 11 | 34 | 27 | N/N | 0 | 0 | 0 | 0 |
| 12 | 33 | 35 | N/N | 0 | 0 | 0 | 0 |
| 13 | 43 | 27 | N/N | 0 | 0 | 0 | 0 |
| 14 | 41 | 33 | N/N | 0 | 0 | 0 | 0 |
| 15 | 25 | 30 | N/N | 0 | 0 | 0 | 0 |
| 16 | 35 | 31 | N/N | 0 | 0 | 0 | 0 |
| 17 | 30 | 32 | N/N | 0 | 0 | 0 | 0 |
| 18 | 32 | 35 | N/N | 0 | 0 | 0 | 0 |
| 19 | 30 | 33 | N/N | 0 | 0 | 0 | 0 |
| 20 | 32 | 28 | N/N | 0 | 0 | 0 | 0 |
| 21 | 33 | 32 | N/N | 0 | 0 | 0 | 0 |
| 22 | 34 | 33 | N/N | 0 | 0 | 0 | 0 |
| 23 | 29 | 30 | N/N | 0 | 0 | 0 | 0 |
| 24 | 30 | 27 | N/N | 0 | 0 | 2 | 0 |
| 25 | 29 | 30 | N/N | 0 | 0 | 0 | 0 |
| 26 | 30 | 28 | N/N^{e} | 0^{f} | 0 | 0 | 0 |
| 27 | 52 | 30 | N/N | 0 | 0 | 0 | 0 |
| 28 | 35 | 35 | N/N | 0 | 0 | 0 | 0 |
| 29 | 30 | 27 | N/N | 0 | 0 | 0 | 0 |
| 30 | 50 | 26 | N/N | 0 | 0 | 1 | 0 |
| 31 | 35 | 31 | N/N | 0 | 0 | 0 | 0 |
| 32 | 35 | 34 | N/N | 0 | 0 | 0 | 0 |
| 33 | 20^{g} | 30 | N/N | 0 | 0 | 0 | 0 |
| 34 | 31 | 30 | N/N | 0 | 0 | 0 | 0 |
| 35 | 32 | 28 | N/N | 0 | 0 | 0 | 0 |
| 36 | 30 | 37 | N/N | 0 | 0 | 0 | 0 |
| 37 | 35 | 38 | N/N | 0 | 0 | 0 | 0 |
| 38 | 34 | 32 | N/N | 0 | 0 | 0 | 0 |
| 39 | 35 | 30 | N/N | 0 | 0 | 0 | 0 |
| 40 | 31 | 32 | N/N | 0 | 0 | 0 | 0 |
| Pos./Tot.^{h} | 3/40 | 1/40 | 0/40/0/40 | 0/40 | 0/40 | 2/40 | 0/40 |

^{a}Clinical Signs
^{b}Post Challenge
^{c}Morbidity (Depression and/or Ruffled Feathers)/Mortality
^{d}Bone Marrow
^{e}Negative/Negative
^{f}0 = Normal; 1 = Slight; 2 = Moderate; 3 = Severe
^{g}Hematocrit Values of ≦25 = Anemia
^{h}Number Positive/Total

TABLE 22
Test 3 Hematocrit Values, Clinical Signs and CIA Gross Lesion Scores of
Challenged Chicks from Non-Vaccinated Breeders.

| | Hematocrit Values | | Clin. Signs^{a} | Gross Lesion Scores | | | |
|---|---|---|---|---|---|---|---|
| Bird No. | 14 Day pc^{b} | 21 Day pc | Mor./Mort.^{c} | Liver | BM^{d} | Thymus | Muscle |
| 1 | 19^{e} | ND^{f} | N/P^{g} | 2^{h} | 2 | 3 | 2 |
| 2 | 22 | 32 | N/N | 0 | 0 | 0 | 0 |
| 3 | 50 | ND | P/P | 0 | 0 | 3 | 0 |
| 4 | 32 | 28 | N/N | 0 | 0 | 2 | 0 |
| 5 | 31 | 27 | N/N | 0 | 2 | 0 | 0 |
| 6 | 32 | 29 | N/N | 0 | 0 | 0 | 0 |
| 7 | 26 | 19 | N/N | 0 | 0 | 2 | 0 |
| 8 | 30 | 27 | N/N | 0 | 0 | 0 | 0 |
| 9 | 23 | ND | P/P | 0 | 2 | 2 | 0 |
| 10 | 17 | 29 | N/N | 0 | 0 | 0 | 0 |
| 11 | 23 | 35 | N/N | 0 | 0 | 0 | 0 |
| 12 | 20 | ND | N/P | 0 | 3 | 3 | 0 |
| 13 | 18 | ND | P/P | 0 | 0 | 2 | 0 |
| 14 | 22 | ND | N/P | 0 | 2 | 3 | 1 |
| 15 | 44 | 13 | N/N | 0 | 2 | 2 | 0 |
| 16 | 30 | 32 | N/N | 0 | 0 | 1 | 0 |
| 17 | 14 | ND | N/P | 0 | 0 | 3 | 0 |
| 18 | 31 | 26 | N/N | 0 | 0 | 1 | 0 |
| 19 | 20 | ND | N/P | 0 | 2 | 3 | 0 |
| 20 | 23 | 10 | N/N | 0 | 2 | 2 | 0 |
| 21 | 33 | 20 | N/N | 0 | 0 | 2 | 0 |
| 22 | 23 | ND | P/P | 0 | 0 | 3 | 0 |
| 23 | 22 | ND | N/P | 0 | 0 | 3 | 0 |
| 24 | 29 | 27 | N/N | 0 | 2 | 2 | 0 |
| 25 | 30 | 15 | N/N | 0 | 0 | 1 | 0 |
| 26 | 29 | 35 | N/IN | 0^{h} | 0 | 0 | 0 |
| 27 | 24^{e} | 33 | N/N | 0 | 0 | 0 | 0 |
| 28 | 27 | 20 | N/N | 0 | 0 | 0 | 0 |
| 29 | 32 | 19 | N/N | 0 | 2 | 1 | 0 |
| 30 | 25 | ND^{f} | P/P | 2 | 0 | 3 | 1 |
| 31 | 22 | 18 | N/N | 0 | 1 | 2 | 0 |
| 32 | 33 | 34 | N/N | 0 | 0 | 0 | 0 |
| 33 | 23 | ND | N/P^{g} | 0 | 2 | 3 | 0 |
| 34 | 25 | 35 | N/N | 0 | 0 | 0 | 0 |
| 35 | 16 | ND | N/P | 0 | 0 | 3 | 0 |
| 36 | 28 | 15 | N/N | 0 | 0 | 0 | 0 |
| 37 | 29 | 25 | N/N | 0 | 0 | 0 | 0 |
| 38 | 30 | 32 | N/N | 0 | 0 | 0 | 0 |
| 39 | 29 | 25 | N/N | 0 | 0 | 2 | 0 |
| 40 | 31 | 23 | N/N | 0 | 0 | 0 | 0 |
| Pos./Tot.ⁱ | 19/40 | 12/27 | 5/40 13/40 | 2/40 | 12/40 | 25/40 | 3/40 |

^{a}Clinical Signs
^{b}Post Challenge
^{c}Morbidity (Depression and/or Ruffled Feathers)/Mortality
^{d}Bone Marrow
^{e}Hematocrit Values ≦25 = Anemia
^{f}Not Done
^{g}Negative/Positive (CIAV Associated Mortality)
^{h}0 = Normal; 1 = Slight; 2 = Moderate; 3 = Severe
ⁱNumber Positive/Total

TABLE 22
Test 3 Hematocrit Values, Clinical Signs and CIA Gross Lesion Scores of
Chicks from Non-Vaccinated Breeders; Not Challenged.

| | Hematocrit | | | | | | |
|---|---|---|---|---|---|---|---|
| | Values | | | | | | |
| | 14 | 21 | | | | | |
| | Day | Day | Clin. Signs^{a} | Gross Lesion Scores | | | |
| Bird No. | pc^{b} | pc | Mor./Mort.^{c} | Liver | BM^{d} | Thymus | Muscle |
| 1 | 35 | 34 | N/N^{e} | O^{f} | 0 | 0 | 0 |
| 2 | 39 | 35 | N N | 0 | 0 | 0 | 0 |
| 3 | 33 | 34 | N/N | 0 | 0 | 0 | 0 |
| 4 | 37 | 35 | N/N | 0 | 0 | 0 | 0 |
| 5 | 38 | 33 | N/N | 0 | 0 | 0 | 0 |
| 6 | 32 | 35 | N/N | 0 | 0 | 0 | 0 |
| 7 | 35 | 37 | N/N | 0 | 0 | 0 | 0 |
| 8 | 29 | 39 | N/N | 0 | 0 | 0 | 0 |
| 9 | 35 | 36 | N/N | 0 | 0 | 0 | 0 |
| 10 | 32 | 37 | N/N | 0 | 0 | 0 | 0 |
| 11 | 33 | 38 | N/N | 0 | 0 | 0 | 0 |
| 12 | 33 | 34 | N/N | 0 | 0 | 0 | 0 |
| 13 | 31 | 35 | N/N | 0 | 0 | 0 | 0 |
| 14 | 30 | 35 | N/N | 0 | 0 | 0 | 0 |
| 15 | 36 | 40 | N/N | 0 | 0 | 0 | 0 |
| 16 | 30 | 39 | N/N | 0 | 0 | 0 | 0 |
| 17 | 30 | 38 | N/N | 0 | 0 | 0 | 0 |
| 18 | 30 | 36 | N/N | 0 | 0 | 0 | 0 |
| 19 | 40 | 35 | N/N | 0 | 0 | 0 | 0 |
| 20 | 35 | 35 | N/N | 0 | 0 | 0 | 0 |
| 21 | 35 | 35 | N/N | 0 | 0 | 0 | 0 |
| 22 | 35 | 33 | N/N | 0 | 0 | 0 | 0 |
| 23 | 34 | 41 | N/N | 0 | 0 | 0 | 0 |
| 24 | 28 | 41 | N/N | 0 | 0 | 0 | 0 |
| 25 | 32 | 38 | N/N | 0 | 0 | 0 | 0 |
| Pos./Tot.^{g} | 0/25 | 0/25 | 0/25/0/25 | 0/25 | 0/25 | 0/25 | 0/25 |

^{a}Clinical Signs
^{b}Post Challenge
^{c}Morbidity (Depression and/or Ruffled Feathers)/Mortality
^{d}Bone Marow
^{e}Negative/Negative
^{f}0 = Normal; 1 = Slight; 2 = Moderate; 3 = Severe
^{g}Number Positive/Total

TABLE 23
Summary of Test 3 Hematocrit, Morbidity, Mortality and Gross
Lesion of Challenged and Non-Challenged Chicks.

| Test Group | Hematocrit | Morbidity | Mortality | PM^{a} |
|---|---|---|---|---|
| CIAV | 4/40 (10%)^{c} | 0/40 | 0/40 | 2/40 (5%)^{d} |
| Vaccinated^{b} | | | | |
| Non- | 29/40 (73%) | 5/40 (13%) | 13/40 (33%) | 26/40 (65%) |
| Vaccinated^{b} | | | | |
| Negative | 0/25 | 0/25 | 0/25 | 0/25 |
| Control | | | | |

^{a}Post-Mortem CIA Gross Lesion Scores
^{b}Challenge Group
^{c}Number Positive/Total
^{d}Positive Chicks = Gross Lesion Scores ≧1

TABLE 24
Statistical Evaluation of Test 3 Hematocrit Values and Clinical Signs of
Challenged Chicks using Fisher's Exact Probability Test

| | Hematocrit | | | | |
|---|---|---|---|---|---|
| | Values | | | | |
| | 14 | 21 | Clinical Signs | | |
| Test Group | Day pc^{a} | Day pc | Morbidity | Mortality | Combined |
| CIAV Vaccinated | 3/40 | 1/40 | 0/40 | 0/40 | 4/40^{b} |
| Non-Vaccinated | 19/40 | 12/27 | 5/40 | 13/40 | 30/40 |
| p value | <0.001 | <0.001 | 0.027 | <0.001 | <0.001 |

^{a}Post Challenge
^{b}Combined Hematocrit Values and Clinical Signs

TABLE 25
Statistical Evaluation of Test 3 CIA Gross lesion Scores of
Challenged Chicks from Vaccinated and Non-Vaccinated
Breeders using the Mann-Whitney Test

| | Gross Lesion Scores^{a} | | | | |
|---|---|---|---|---|---|
| | Liver | BM^{b} | Thymus | Muscle | Combined^{c} |
| p value | 0.7 | 0.021 | <0.001 | 0.5637 | <0.001 |

^{a}Raw Data Found in Tables 8 and 9
^{b}Bone Marrow
^{c}Combined Gross Lesion Scores

### Example 7

### Efficacy of a Chicken Anemia Virus Vaccine Evaluated by Maternal Antibody Protection of Progeny from Chickens 27 and 37 Weeks Following Drinking Water Administration of the Vaccine

Host animal efficacy and duration of immunity studies were conducted in chickens by challenge of day-old progeny hatched from 27 and 37 week-old hens, which were previously vaccinated with chicken infectious anemia virus, Del Ros strain (CIAV-DR) vaccine at 9 weeks of age by drinking water. The challenge procedure of progeny and parameters of measurement of efficacy by maternal antibody protection (passive immunity) provided by hens vaccinated in the drinking water were the same as for chicken anemia virus vaccine administered by the wing web route (see Example 6).

Progeny were hatched from fertile eggs laid 18 and 28 weeks post vaccination when hens were 27 and 37 weeks of age, respectively. Intra-abdominal challenge of day-old progeny was used to evaluate maternal antibody protection provided by CLAV-DR following drinking water vaccination of CIAV-negative SPF chickens at 9 weeks of age. Post challenge observations of progeny through 21 days of age included clinical signs, hematocrit value determinations and post-mortem examinations for gross lesions characteristic of chicken infectious anemia (CIA).

Chickens used for vaccination in this study were CIAV negative, SPF leghorn-type purchased from SPAFAS, Ric. Birds were wing banded for identification upon arrival. Twnety randomly selected chickens at 9 weeks of age were bled for CIAV serology to confirm negative serological status using the IDEXX ELISA CIAV kit. On the same day, 40 females and 5 males designated as vaccinates were water starved and then permitted to drink water containing CIAV-DR vaccine. The average of five replicate titers of the CIAV vaccine conducted after vaccination in MSB-1 cells determined a dose contained 10^{5.5} TCID₅₀. Negative control breeder chickens fiom the same source and hatch date were maintained. Two efficacy/duration of iimnunity studies identified as Study 1 and Study 2 were conducted on progeny from 27 and 37 week-old hens, respectively Chicks were challenged at one day of age with CIAV. The challenge virus was liver homogenate extract derived firom chicks inoculated with a Texas field isolate of CIAV. Each chick was inoculated intra-abdominally with approximately 10^{2.6} CID₅₀ per 0.2 ml.

Each study consisted of a group of progeny from non-vaccinated hens maintained as non-challenged negative controls, a group of CIAV challenged progeny from non-vaccinated hens that served as positive controls, and a group of CIAV challenged progeny from vaccinated hens. Chicks of all treatment groups were maintained in filtered air, negative pressure isolation units and observed through 21 days for depression, ruffled feathers and mortality. Blood samples were collected from all chicks at 14 and 21 days post challenge (dpc) for hematocrit value detenninations as a measure of anemia. The procedure used for determining hematocrit values was that of Rosenberger and Cloud (Avian Dis. 33:753-759, 1989). A chick with a hematocrit value of ≦25 was considered to be anemic. Additionally, chicks of all treatments were examined at 21 dpc for gross lesions characteristic of CIA including pale bone marrow, swelling and discoloration of the liver and spleen, and hemorrhage lesions in the skin and muscles. Treatment comparisons were based on the number of individuals within a treatment (per total examined) exhibiting specific gross lesions of CIA. Data were statistically analyzed using Fisher's Exact Probability Test and Mann-Whitney Test.

Serological pre-vaccination serum samples using the IDEXX ELISA kit confirmed the CIAV negative status of the 9-week-old chickens acquired from SPAFAS, Inc. that were used in this study. ELISA results are given in Table 26.

Results of the two studies reported herein demonstrated that 10^{5.5} TCID₅₀ of CIAV-DR vaccine administered by drinking water to 9-week-old pullets significantly protected progeny at p<0.05 through 37 weeks of age (i.e. 28 weeks post vaccination) when compared to progeny from non-vaccinated hens. A gross lesion score ≧1 for any one of the tissues examined (i.e. liver, bone marrow, thymus and muscle) was recorded as a CIA positive chick. There was a significant difference at p<0.05 in progeny of vaccinated hens compared to non-vaccinated hens in Study 1 and Study 2 against morbidity and mortality, anemia, and gross lesions characteristic of CIA.

### Results of Study 1

Forty day-old chicks from non-vaccinated breeders challenged with CIAV were evaluated in this study as the positive control group. The death of one of 25 chicks from the non-challenged negative control group occurred early in the test period and could not be attributed to any specific cause. Twenty-four negative controls remained for evaluation. A power outage in the isolator holding 40 challenged chicks from the CIAV vaccinated hens at 3 dpc and resulted in the death of 15 of the 40 chicks leaving 25 chicks of this treatment group for evaluation in this study (See Table 29). One chick from the CIAV vaccinated group died at 5 dpc. The chick had no gross lesions or clinical signs of CIAV. Therefore, mortality was ruled due to non-CIAV related causes.

The 24 non-challenged negative control chicks did not exhibit morbidity, mortality or gross lesions of CIA. One of 22 serum samples collected from chicks at 21 dpc had a hematocrit value of 23, but the chick had no other characteristic sign of CIA. Results are given in Table 27.

The challenge procedure induced CIA in progeny from non-vaccinated breeders. Hematocrit values ≦25 at either 14 or 21 dpc were demonstrated in 36 of 40 (90%) positive control chicks. Morbidity was noted in 5 of 40 (12.5%) chicks, whereas, mortality was experienced in 10 of 40 (25%) chicks. Gross lesions were evident in 33 of 40 (82.5%) chicks. Results are given in Table 28.

Statistical evaluations by Fisher's Exact Probability Test of hematocrit values demonstrated that there was a significant difference at p<0.001 against anemia, a significant difference at p=0.012 against combined morbidity and mortality, and a significant difference of p<0.001 in the number of birds with CIA gross lesion scores in progeny from vaccinated breeders compared to progeny from non-vaccinated breeders. Statistical analysis of gross lesion scores by Mann-Whitney Test demonstrated a significant difference of p<0.001 in the bone marrow and the thymus. There was a significant difference at p<0.001 by Fisher's Exact Test of the number of birds with gross lesions of progeny from vaccinated breeders compared to progeny from non-vaccinated breeders. Results and statistical evaluations given in Tables 29, 30, 31 and 32.

### Results of Study 2

The groups of study 2 consisted of non-challenged negative controls from non-vaccinated hens (n=25), CIAV challenged controls from non-vaccinated hens (n=40) and CIAV challenged progeny from 37-week-old CIAV vaccinated breeder hens (n=40). Throughout the 21-day test, negative control chickens remained free of anemia as determined by hematocrit values, morbidity, mortality and gross lesion scores associated with CIA. Results are given in Table 33.

The CIAV positive control chicks exhibited lowered hematocrit values, clinical signs and gross lesions typical of CIA. Hematocrit values ≦25 at either 14 or 21 dpc were demonstrated in 32 of 39 (82.1%) positive control chicks. Morbidity was noted in 6 of 40 (15.0%) chicks, and mortality was experienced in 12 of 40 (30.0%) chicks. Gross lesions were evident at post mortem in 24/40 (60.0%) of chicks. Results are given in Table 34.

Following CIAV challenge a significant difference at p<0.05 was demonstrated in progeny from CIAV vaccinated hens compared to progeny from non-vaccinated hens in hematocrit values at 14 and 21 dpc, in morbidity and mortality, and in gross lesions scores. Fisher's Exact Probability Test of hematocrit values demonstrated a significant difference at p<0.001 against anemia, a significant difference at p<0.001 against morbidity and mortality, and a significant difference at p<0.001 in the number of birds with CIA gross lesions scores. Results and statistical evaluations are given in Tables 10, 11, 12 and 13. Please note that one chick from the CIAV vaccinated group died 3 dpc and another at 8 dpc. The chicks had no gross lesions or clinical signs of CIAV. Therefore, mortality was ruled due to non-CIAV related causes.

These studies demonstrated that CIAV matemal antibody provided significant protection against CIA at p<0.05 to progeny of SPF white leghorn type chickens, which were previously vaccinated at 9 weeks of age with the live chicken infectious anemia virus vaccine administrated via the drinking water. The protection was assessed on the basis of clinical signs, morbidity/mortality, and CIAV specific lesions at necropsy. These studies demonstrated that maternal antibody protection was provided to chicks by hens through at least 37 weeks of age (28 weeks post vaccination).

TABLE 26
Pre-vaccination Serological Results by ELISA of 9-week-old
SPF Chickens to Confirm Negative Serological Status Prior
to Vaccination with Water-administered CIAV Vaccine.

| | Band | S/N Ratio | CIAV Serological |
|---|---|---|---|
| Bird No. | No. | by ELISA | Status |
| 1 | 104 | 0.89 | Negative^{a} |
| 2 | 108 | 0.90 | Negative |
| 3 | 128 | 1.00 | Negative |
| 4 | 133 | 0.95 | Negative |
| 5 | 141 | 0.98 | Negative |
| 6 | 190 | 0.84 | Negative |
| 7 | 191 | 0.95 | Negative |
| 8 | 201 | 0.89 | Negative |
| 9 | 215 | 1.00 | Negative |
| 10 | 217 | 0.85 | Negative |
| 11 | 742 | 0.91 | Negative |
| 12 | 747 | 0.89 | Negative |
| 13 | 753 | 0.82 | Negative |
| 14 | 765 | 0.91 | Negative |
| 15 | 768 | 0.82 | Negative |
| 16 | 826 | 0.97 | Negative |
| 17 | 838 | 0.89 | Negative |
| 18 | 850 | 0.91 | Negative |
| 19 | 856 | 0.86 | Negative |
| 20 | 866 | 0.98 | Negative |

^{a}Negative = S/N Ratio > 0.6 (IDEXX Kit interpretation)

TABLE 27
Study 1 Hematocrit Values, Clinical Signs and CIA Gross Lesion
Scores of Non-challenged Chicks from 27-Week-old
Non-Vaccinated Breeder Chickens (Negative Controls).

| | Hematocrit | | Clinical | | | | |
|---|---|---|---|---|---|---|---|
| | Values | | Signs | Gross Lesion Scores | | | |
| | 14 | 21 | Morbidity/ | | Bone | | |
| Bird No. | dpc^{a} | dpc | Mortality | Liver | Marrow | Thymus | Muscle |
| 1 | NS^{b} | 30 | N/N^{c} | 0^{d} | 0 | 0 | 0 |
| 2 | 37 | 32 | N/N | 0 | 0 | 0 | 0 |
| 3 | 31 | 39 | N/N | 0 | 0 | 0 | 0 |
| 4 | 30 | 27 | N/N | 0 | 0 | 0 | 0 |
| 5 | 32 | 37 | N/N | 0 | 0 | 0 | 0 |
| 7 | 33 | 33 | N/N | 0 | 0 | 0 | 0 |
| 8 | 37 | NS | N/N | 0 | 0 | 0 | 0 |
| 9 | 27 | 30 | N/N | 0 | 0 | 0 | 0 |
| 10 | 28 | 32 | N/N | 0 | 0 | 0 | 0 |
| 11 | 34 | 27 | N/N | 0 | 0 | 0 | 0 |
| 12 | 31 | 40 | N/N | 0 | 0 | 0 | 0 |
| 13 | 34 | 26 | N/N | 0 | 0 | 0 | 0 |
| 14 | 26 | 26 | N/N | 0 | 0 | 0 | 0 |
| 15 | 28 | 31 | N/N | 0 | 0 | 0 | 0 |
| 16 | 32 | 33 | N/N | 0 | 0 | 0 | 0 |
| 17 | 35 | 34 | N/N | 0 | 0 | 0 | 0 |
| 18 | 32 | 29 | N/N | 0 | 0 | 0 | 0 |
| 19 | 35 | 32 | N/N | 0 | 0 | 0 | 0 |
| 20 | NS | 34 | N/N | 0 | 0 | 0 | 0 |
| 21 | 35 | 33 | N/N | 0 | 0 | 0 | 0 |
| 22 | 28 | 23 | N/N | 0 | 0 | 0 | 0 |
| 23 | 33 | 38 | N/N | 0 | 0 | 0 | 0 |
| 24 | NS | 27 | N/N | 0 | 0 | 0 | 0 |
| 25 | 31 | NS | N/N | 0 | 0 | 0 | 0 |
| No. | 0/21 | 1/22 | 0/24/0/24 | 0/24 | 0/24 | 0/24 | 0/24 |
| Positive | | | | | | | |

^{a}Days post challenge
^{b}No sample
^{c}N = negative
^{d}0 = normal, 1 = slight, 2 = moderate, 3 = severe gross lesions associated with CIA

TABLE 28
Study 1 Hematocrit Values, Clinical Signs, Mortality and CIA Gross
Lesion Scores of Chicks from 27-week-old Non-vaccinated Breeder
Chickens Challenged at Day of Age with CIAV (Positive Controls).

| | Hematocrit | | Clinical | | | | |
|---|---|---|---|---|---|---|---|
| | Values | | Signs | Gross Lesion Scores | | | |
| | 14 | 21 | Morbidity/ | | Bone | | |
| Bird No. | dpc^{a} | dpc | Mortality | Liver | Marrow | Thymus | Muscle |
| 1 | 16^{b} | ND^{c} | N^{d}/P^{e} | 1^{f} | 3 | 3 | 2 |
| 2 | 12 | ND | N/P | 0 | 3 | 3 | 0 |
| 3 | 25 | 20 | N/N | 0 | 0 | 1 | 0 |
| 4 | 30 | 17 | N/N | 0 | 2 | 0 | 0 |
| 5 | 18 | 6 | P/N | 1 | 3 | 3 | 1 |
| 6 | 10 | ND | N/P | 0 | 3 | 3 | 2 |
| 7 | 28 | 13 | P/N | 1 | 2 | 2 | 0 |
| 8 | 33 | 24 | N/N | 0 | 2 | 2 | 0 |
| 9 | 22 | ND | N/P | 0 | 3 | 3 | 0 |
| 10 | 25 | 10 | N/N | 0 | 2 | 3 | 1 |
| 11 | 27 | 32 | N/N | 0 | 0 | 0 | 0 |
| 12 | 30 | 13 | N/N | 0 | 2 | 3 | 0 |
| 13 | NS^{g} | 15 | N/N | 0 | 3 | 1 | 0 |
| 14 | 20 | 29 | N/N | 0 | 0 | 0 | 0 |
| 15 | 17 | ND | N/P | 0 | 3 | 3 | 0 |
| 16 | 16 | 30 | N/N | 0 | 0 | 0 | 0 |
| 17 | 25 | 11 | P/N | 0 | 3 | 3 | 2 |
| 18 | 23 | 25 | N/N | 0 | 1 | 1 | 0 |
| 19 | 33 | 15 | N/N | 0 | 2 | 2 | 0 |
| 20 | 15 | ND | N/P | 0 | 2 | 2 | 0 |
| 21 | 29 | 16 | N/N | 0 | 2 | 2 | 0 |
| 22 | 25 | 31 | N/N | 0 | 0 | 0 | 0 |
| 23 | 23 | 13 | P/N | 2 | 3 | 3 | 2 |
| 24 | 44 | 20 | N/N | 0 | 2 | 1 | 0 |
| 25 | 25 | 21 | N/N | 0 | 0 | 2 | 0 |
| 26 | 35 | 23 | N/N | 0 | 0 | 1 | 0 |
| 27 | 12 | 20 | N/N | 0 | 2 | 2 | 1 |
| 28 | 30 | 24 | N/N | 0 | 0 | 0 | 0 |
| 29 | 33 | 18 | N/N | 0 | 2 | 2 | 0 |
| 30 | 25 | 39 | N/N | 0 | 0 | 0 | 0 |
| 31 | 25 | ND | P/P | 0 | 2 | 2 | 1 |
| 32 | 22 | ND | N/P | 0 | 1 | 2 | 0 |
| 33 | 26 | ND | N/P | 0 | 3 | 3 | 0 |
| 34 | 20 | 25 | N/N | 0 | 1 | 1 | 0 |
| 35 | 17 | 15 | N/N | 0 | 1 | 2 | 0 |
| 36 | 33 | 28 | N/N | 0 | 0 | 0 | 0 |
| 37 | 31 | 15 | N/N | 0 | 2 | 2 | 0 |
| 38 | 25 | ND | N/P | 0 | 0 | 2 | 0 |
| 39 | NS | 27 | N/N | 0 | 0 | 1 | 0 |
| 40 | 30 | 24 | N/N | 0 | 1 | 0 | 0 |
| No. | 23/38 | 23/30 | 5/40/ | 4/40 | 28/40 | 31/40 | 8/40 |
| Positive | | | 10/40 | | | | |

| | | | |
|---|---|---|---|
| No. Birds with | No. Dead or | No. Birds with | No. Birds |
| CIA Positive | Morbid = 14/40 | CIA Gross | Positive for |
| Hematocrit | (35.0%) | Lesion Scores | CIA/Total = |
| Values^{b}/Total = | | ≧1/Total = | 38/40 (95.0%) |
| 36/40 (90.0%) | | 33/40 (82.5%) | |

^{a}Days post challenge
^{b}Hematocrit values ≦25 = anemia
^{c}Not Done
^{d}N = negative
^{e}positive for clinical signs or CIAV mortality
^{f}0 = normal, 1 = slight, 2 = moderate, 3 = severe gross lesions associated with CIA
^{g}No sample

TABLE 29
Study 1 Hematocrit Values, Clinical Signs, Mortality and CIA Gross
Lesion Scores of Chicks with Maternal Antibody from 27-Week-old CIAV
Vaccinated Breeder Chickens Challenged at Day of Age with CIAV.

| | Hematocrit | | Clinical | | | | |
|---|---|---|---|---|---|---|---|
| | Values | | Signs | Gross Lesion Scores | | | |
| | 14 | 21 | Morbidity/ | | Bone | | |
| Bird No. | dpc^{a} | dpc | Mortality | Liver | Marrow | Thymus | Muscle |
| 1 | 23^{b} | 32 | N^{c}/N | 0 | 0 | 0 | 0 |
| 5 | ND^{d} | ND | N/Q^{e} | 0 | 0 | 0 | 0 |
| 6 | 38 | 27 | N/N | 0 | 0 | 0 | 0 |
| 7 | 33 | 28 | N/N | 0 | 0 | 0 | 0 |
| 9 | 29 | 30 | N/N | 0 | 0 | 0 | 0 |
| 10 | 29 | 30 | N/N | 0 | 0 | 0 | 0 |
| 11 | 30 | 30 | N/N | 0 | 0 | 0 | 0 |
| 12 | 29 | 29 | N/N | 0 | 0 | 0 | 0 |
| 13 | 27 | 39 | N/N | 0 | 0 | 0 | 0 |
| 14 | 34 | 34 | N/N | 0 | 0 | 0 | 0 |
| 19 | 28 | 26 | N/N | 0 | 0 | 0 | 0 |
| 20 | 31 | 39 | N/N | 0 | 0 | 0 | 0 |
| 21 | 30 | NS^{f} | N/N | 0 | 2^{g} | 2 | 0 |
| 23 | 34 | 34 | N/N | 0 | 0 | 0 | 0 |
| 24 | 35 | 28 | N/N | 0 | 0 | 0 | 0 |
| 25 | 30 | 26 | N/N | 0 | 0 | 0 | 0 |
| 28 | 30 | 27 | N/N | 0 | 0 | 0 | 0 |
| 29 | 29 | 28 | N/N | 0 | 0 | 0 | 0 |
| 30 | 35 | 35 | N/N | 0 | 0 | 0 | 0 |
| 33 | 32 | 33 | N/N | 0 | 0 | 0 | 0 |
| 34 | 27 | 35 | N/N | 0 | 0 | 0 | 0 |
| 35 | 23 | 24 | N/N | 0 | 2 | 2 | 0 |
| 38 | 16 | NS | N/P^{h} | 0 | 3 | 2 | 2 |
| 39 | 15 | 33 | N/N | 0 | 0 | 0 | 0 |
| 40 | 27 | 35 | N/N | 0 | 0 | 0 | 0 |
| No. | 4/24 | 1/22 | 0/25 2/25 | 0/25 | 3/25 | 3/25 | 1/25 |
| Positive | | | | | | | |

| | | | |
|---|---|---|---|
| No. Birds with CIA | No. Dead | No. Birds with | No. Birds |
| Positive Hematocrit | or Morbid | CIA Gross | CIA Positive/ |
| Values^{b}/Total = 4/24 | 2/25 | Lesion Scores | Total = 5/25 |
| (16.7%) | (8.0%) | ≧1/Total = | (20.0%) |
| | | 3/25 (12.0%) | |

^{a}Days post challenge
^{b}Hematocrit values ≦25 = anemia
^{c}N = negative
^{d}Not done
^{e}Q = non CIAV associated mortality
^{f}No serum
^{g}0 = normal, 1 = slight, 2 = moderate, 3 = severe gross lesions associated with CIA
^{h}P = positive for clinical signs or CIAV mortality

TABLE 30
Study 1 Summary of Hematocrit Values of CIAV Challenged
Chicks from 27-Week-old Vaccinated and
Non-vaccinated Breeder Chickens.

| | No. Birds with | No. Birds with | No. Birds with |
|---|---|---|---|
| | Hematocrit ≦25 | Hematocrit ≦25 | Hematocrit ≦25 |
| | at 14 dpc/ | at 21 dpc/ | at either 14 or |
| | Total No. | Total No. | 21 dpc/Total No. |
| Group | Evaluated | Evaluated | Evaluated |
| Negative | 0/21 | 1/22 | 1/24 |
| Control | | (4.5%) | (4.2%) |
| Positive | 23/38 | 23/30 | 36/40 |
| Control | (60.5%) | (76.7%) | (90.0%) |
| Progeny from | 4/24 | 1/22 | 4/24 |
| CIAV | (16.7%) | (4.5%) | (16.7%) |
| Vaccinated | | | |
| Hens | | | |
| Fisher's Exact | <0.001^{a} | <0.001^{a} | <0.001^{a} |
| Test p = | | | |

^{a}Statistical difference by Fisher's Exact Test at p < 0.001 between positive controls and progeny from CIAV vaccinated breeder chickens.

TABLE 31
Study 1 Summary of Clinical Signs and Mortality of CIAV
Challenged Chicks from 27 Week-old Vaccinated
and Non-vaccinated Breeder Chickens.

| | | | No. with Clinical |
|---|---|---|---|
| | No. with Clinical | No. | Signs or |
| Group | Signs/Total | Dead/Total | Dead/Total |
| Negative | 0/24 | 0/24 | 0/24 |
| Control | | | |
| Positive | 5/40 | 10/40 | 14/40 |
| Control | (12.5%) | (25.0%) | (35.0%) |
| Progeny from | 0/25 | 2/25 | 2/25 |
| CIAV Vaccinated | | (8.0%) | (8.0%) |
| Hens | | | |
| Fisher's Exact | 0.08 | 0.08 | 0.012^{a} |
| Test p = | | | |

^{a}Statistical difference by Fisher's Exact Test at p < 0.05 between positive control group and progeny from CIAV vaccinated breeder chickens.

TABLE 32
Study 1 Summary of CIA Gross Lesion Scores of CIAV Challenged
Progeny from 27-Week-old CIAV Vaccinated and Non-vaccinated
Breeder Chickens.

| | No. Birds with Gross Lesion | | | | |
|---|---|---|---|---|---|
| | Scores ≧1 (GLS)/Total No. | | | | |
| | Birds at Post-mortem | | | | No Birds |
| | | Bone | | | with GLS |
| Group | Liver | Marrow | Thymus | Muscle | ≧1/Total |
| Negative | 0/24 | 0/24 | 0/24 | 0/24 | 0/24 |
| Control | | | | | |
| Positive | 4/40 | 28/40 | 31/40 | 8/40 | 33/40 |
| Control | (10.0%) | (70.0%) | (77.5%) | (20.0%) | (82.5%) |
| Progeny from CIAV | 0/25 | 3/25 | 3/25 | 1/25 | 3/25 |
| Vaccinated Hens | | (12.0%) | (12.0%) | (4.0%) | (12.0%) |
| Mann-Whitney Test | 0.5 | <0.001^{a} | <0.001^{a} | 0.29 | <0.001^{a} |
| p = | | | | | |
| Fisher's Exact Test p = | NA^{b} | NA | NA | NA | <0.001^{c} |

^{a}Statistical difference at p < 0.001 between positive control group and progeny from CIAV vaccinated hens by Mann-Whitney Test.
^{b}Not applicable.
^{c}Statistical difference at p < 0.001 between positive control group and progeny from CIAV vaccinated hens by Fisher's Exact Test.

TABLE 33
Study 2 Hematocrit Values, Clinical Signs, Mortality and CIA
Gross Lesion Scores of Non-challenged Chicks from 37-Week-old
Non-Vaccinated Breeder Chickens (Negative Controls).

| | Hematocrit | | Clinical | | | | |
|---|---|---|---|---|---|---|---|
| | Values | | Signs | Gross Lesion Scores | | | |
| Bird | 14 | | Morbidity/ | | Bone | | |
| No. | dpc^{a} | 21 dpc | Mortality | Liver | Marrow | Thymus | Muscle |
| 1 | 31 | 34 | N/N^{b} | 0^{c} | 0 | 0 | 0 |
| 2 | 35 | 33 | N/N | 0 | 0 | 0 | 0 |
| 3 | 35 | 37 | N/N | 0 | 0 | 0 | 0 |
| 4 | NS^{d} | 35 | N/N | 0 | 0 | 0 | 0 |
| 5 | 35 | 34 | N/N | 0 | 0 | 0 | 0 |
| 6 | 34 | 33 | N/N | 0 | 0 | 0 | 0 |
| 7 | 34 | 31 | N/N | 0 | 0 | 0 | 0 |
| 8 | 33 | 34 | N/N | 0 | 0 | 0 | 0 |
| 9 | 35 | 35 | N/N | 0 | 0 | 0 | 0 |
| 10 | 38 | 33 | N/N | 0 | 0 | 0 | 0 |
| 11 | 37 | NS | N/N | 0 | 0 | 0 | 0 |
| 12 | 34 | NS | N/N | 0 | 0 | 0 | 0 |
| 13 | 36 | 35 | N/N | 0 | 0 | 0 | 0 |
| 14 | 38 | 33 | N/N | 0 | 0 | 0 | 0 |
| 15 | 36 | NS | N/N | 0 | 0 | 0 | 0 |
| 16 | NS | 35 | N/N | 0 | 0 | 0 | 0 |
| 17 | 35 | NS | N/N | 0 | 0 | 0 | 0 |
| 18 | 33 | 35 | N/N | 0 | 0 | 0 | 0 |
| 19 | 42 | 38 | N/N | 0 | 0 | 0 | 0 |
| 20 | 32 | 35 | N/N | 0 | 0 | 0 | 0 |
| 21 | 35 | 37 | N/N | 0 | 0 | 0 | 0 |
| 22 | 30 | 31 | N/N | 0 | 0 | 0 | 0 |
| 23 | 35 | 34 | N/N | 0 | 0 | 0 | 0 |
| 24 | 33 | 35 | N/N | 0 | 0 | 0 | 0 |
| 25 | 34 | 34 | N/N | 0 | 0 | 0 | 0 |
| No | 0/23 | 0/21 | 0/25/0/25 | 0/25 | 0/25 | 0/25 | 0/25 |
| positive | | | | | | | |

^{a}Days post challenge
^{b}N = negative
^{c}0 = normal, 1 = slight, 2 = moderate, 3 = severe gross lesions associated with CIA
^{d}No sample

TABLE 34
Study 2 Hematocrit Values, Clinical Signs, Mortality and CIA
Gross Lesion Scores of Chicks from 37-Week-old Non-Vaccinated
Breeder Chickens Challenged at Day of Age with CIAV
(Positive Controls).

| | Hematocrit | | Clinical | | | | |
|---|---|---|---|---|---|---|---|
| | Values | | Signs | Gross Lesion Scores | | | |
| Bird | 14 | | Morbidity/ | | Bone | | |
| No. | dpc^{a} | 21 dpc | Mortality | Liver | Marrow | Thymus | Muscle |
| 1 | 30 | 20 | N/N^{b} | 0 | 0 | 0 | 0 |
| 2 | 25^{c} | 33 | N/N | 0 | 0 | 0 | 0 |
| 3 | NS^{d} | 30 | N/N | 0 | 0 | 0 | 0 |
| 4 | 29 | ND^{e} | N/P^{f} | 0 | 3^{g} | 3 | 0 |
| 5 | 19 | 12 | N/N | 0 | 2 | 1 | 0 |
| 6 | 29 | 23 | N/N | 0 | 2 | 2 | 0 |
| 7 | 27 | 15 | N/N | 0 | 3 | 2 | 0 |
| 8 | 14 | 13 | N/N | 0 | 0 | 3 | 0 |
| 9 | 18 | ND | N/P | 0 | 3 | 3 | 0 |
| 10 | 25 | 20 | N/N | 0 | 1 | 0 | 0 |
| 11 | 33 | 22 | N/N | 0 | 0 | 0 | 0 |
| 12 | 21 | ND | P/P | 0 | 3 | 3 | 0 |
| 13 | 13 | 23 | P/N | 0 | 2 | 0 | 0 |
| 14 | 27 | 23 | N/N | 0 | 0 | 2 | 0 |
| 15 | 20 | ND | N/P | 0 | 3 | 3 | 0 |
| 16 | 22 | ND | N/P | 0 | 2 | 3 | 0 |
| 17 | 24 | ND | N/P | 0 | 3 | 2 | 0 |
| 18 | 20 | 23 | P/N | 0 | 1 | 2 | 0 |
| 19 | 14 | ND | N/P | 0 | 3 | 3 | 0 |
| 20 | 24 | 18 | P/N | 0 | 2 | 3 | 0 |
| 21 | 8 | ND | P/P | 0 | 3 | 3 | 0 |
| 22 | 15 | 16 | N/N | 0 | 3 | 3 | 0 |
| 23 | 24 | 30 | N/N | 0 | 0 | 0 | 0 |
| 24 | 27 | ND | N/P | 0 | 3 | 3 | 0 |
| 25 | 27 | 29 | N/N | 0 | 0 | 0 | 0 |
| 26 | 14 | 15 | N/N | 0 | 2 | 3 | 0 |
| 27 | 23 | ND | N/P | 0 | 3 | 3 | 0 |
| 28 | 13 | 32 | N/N | 0 | 0 | 0 | 0 |
| 29 | 25 | 31 | N/N | 0 | 0 | 0 | 0 |
| 30 | 22 | 28 | N/N | 0 | 0 | 0 | 0 |
| 31 | NS | ND | N/P | 0 | 2 | 3 | 0 |
| 32 | 35 | 30 | N/N | 0 | 0 | 0 | 0 |
| 33 | 24 | 22 | P/N | 0 | 1 | 2 | 0 |
| 34 | 17 | 26 | N/N | 0 | 0 | 0 | 0 |
| 35 | 29 | 29 | N/N | 0 | 0 | 0 | 0 |
| 36 | 25 | 18 | N/N | 0 | 0 | 0 | 0 |
| 37 | 27 | 35 | N/N | 0 | 0 | 0 | 0 |
| 38 | 23 | 30 | N/N | 0 | 0 | 0 | 0 |
| 39 | 20 | 28 | N/N | 0 | 0 | 0 | 0 |
| 40 | 18 | ND | N/P | 0 | 3 | 3 | 0 |
| No. | 27/38 | 15/28 | 6/40/12/40 | 0/40 | 22/40 | 22/40 | 0/40 |
| Positive | | | | | | | |

| | | | |
|---|---|---|---|
| No.Birds with CIA | No. Dead | No. Birds with | No. Birds |
| Positive Hematocrit | or | CIA Gross | Positive for |
| Values^{c}/Total = | Morbid = | Lesion Scores | CIA/Total = |
| 32/39 (82.1%) | 16/40 | ≧1/Total = | 35/40 (87.5%) |
| | (40.0%) | 24/40 (60.0%) | |

^{a}Days post challenge
^{b}N = negative
^{c}Hematocrit values ≦25 = anemia
^{d}No sample
^{e}Not Done
^{f}P = positive for clinical signs or CIAV mortality
^{g}0 = normal, 1 = slight, 2 = moderate, 3 = severe gross lesions associated with CIA

TABLE 35
Study 2 Hematocrit Values, Clinical Signs, Mortality and CIA Gross
Lesion Scores of Chicks with Maternal Antibody from 37-Week-old CIAV
Vaccinated Breeder Chickens Challenged at Day of Age with CIAV.

| | Hematocrit | | Clinical | | | | |
|---|---|---|---|---|---|---|---|
| | Values | | Signs | Gross Lesion Scores | | | |
| Bird | 14 | | Morbidity/ | | Bone | | |
| No. | dpc^{a} | 21 dpc | Mortality | Liver | Marrow | Thymus | Muscle |
| 1 | 30 | NS^{b} | N/NC^{c} | 0 | 0 | 0 | 0 |
| 2 | 31 | 33 | N/N | 0 | 0 | 0 | 0 |
| 3 | 27 | 36 | N/N | 0 | 0 | 0 | 0 |
| 4 | 30 | 36 | N/N | 0 | 0 | 0 | 0 |
| 5 | 33 | ND^{d} | N/P^{e} | 0 | 2^{f} | 3 | 0 |
| 6 | 30 | 40 | N/N | 0 | 0 | 0 | 0 |
| 7 | 27 | 40 | N/N | 0 | 0 | 0 | 0 |
| 8 | 29 | 40 | N/N | 0 | 0 | 0 | 0 |
| 9 | 30 | 32 | N/N | 0 | 0 | 0 | 0 |
| 10 | 28 | 20^{g} | N/N | 0 | 0 | 0 | 0 |
| 11 | 27 | 34 | N/N | 0 | 0 | 0 | 0 |
| 12 | 38 | 39 | N/N | 0 | 0 | 0 | 0 |
| 13 | NS | 38 | N/N | 0 | 0 | 2 | 0 |
| 14 | 20 | 41 | N/N | 0 | 0 | 0 | 0 |
| 15 | 21 | 43 | N/N | 0 | 0 | 0 | 0 |
| 16 | 35 | 40 | N/N | 0 | 0 | 0 | 0 |
| 17 | 31 | 32 | N/N | 0 | 0 | 0 | 0 |
| 18 | 29 | 39 | N/N | 0 | 0 | 0 | 0 |
| 19 | 35 | 46 | N/N | 0 | 0 | 0 | 0 |
| 20 | 23 | 38 | N/N | 0 | 0 | 0 | 0 |
| 21 | 30 | NS | N/N | 0 | 0 | 0 | 0 |
| 22 | 26 | 38 | N/N | 0 | 0 | 0 | 0 |
| 23 | NS | 40 | N/N | 0 | 0 | 0 | 0 |
| 24 | 33 | 43 | N/N | 0 | 0 | 0 | 0 |
| 25 | 42 | 38 | N/N | 0 | 0 | 0 | 0 |
| 26 | 30 | 32 | N/N | 0 | 0 | 0 | 0 |
| 27 | 30 | 40 | N/N | 0 | 0 | 0 | 0 |
| 28 | 25 | 44 | N/N | 0 | 0 | 0 | 0 |
| 29 | 27 | 41 | N/N | 0 | 0 | 0 | 0 |
| 30 | 33 | 35 | N/N | 0 | 0 | 0 | 0 |
| 31 | 36 | 43 | N/N | 0 | 0 | 0 | 0 |
| 32 | 30 | 41 | N/N | 0 | 0 | 0 | 0 |
| 33 | 32 | 38 | N/N | 0 | 0 | 0 | 0 |
| 34 | ND | ND | N/Q^{h} | 0 | 0 | 0 | 0 |
| 35 | 30 | 30 | N/N | 0 | 0 | 0 | 0 |
| 36 | 31 | 41 | N/N | 0 | 0 | 0 | 0 |
| 37 | 42 | 35 | N/N | 0 | 0 | 1 | 0 |
| 38 | 34 | 36 | N/N | 0 | 0 | 0 | 0 |
| 39 | 27 | 35 | N/N | 0 | 0 | 0 | 0 |
| 40 | ND | ND | N/Q^{h} | 0 | 0 | 0 | 0 |
| No. | 4/36 | 1/35 | 0/40/3/40 | 0/40 | 1/40 | 3/40 | 0/40 |
| positive | | | | | | | |

| | | | |
|---|---|---|---|
| No. Birds with | No. Dead or Morbid | No. Birds with | No. Birds CIA |
| CIA Positive | 3/40 (7.5%) | CIA Gross | Positive/Total = |
| Hematocrit | | Lesion Scores | 8/40 (20.0%) |
| Values^{g}/Total = | | ≧1/Total = | |
| 5/38 (13.2%) | | 3/40 (7.5%) | |

^{a}Days post challenge
^{b}No serum
^{c}N = negative
^{d}Not done
^{e}P = positive for clinical signs and CIAV mortality
^{f}0 = normal, 1 = slight, 2 = moderate, 3 = severe gross lesions associated with CIA
^{g}Hematocrit values ≦25 = anemia
^{h}Q = non CIAV associated mortality

TABLE 36
Study 2 Summary of Hematocrit Values of CIAV Challenged Chicks
from 37-week-old Vaccinated and Non-vaccinated Breeder Chickens.

| | No. Birds with | | No. Birds with |
|---|---|---|---|
| | Hematocrit | No. Birds with | Hematocrit ≦25 |
| | ≦25 at | Hematocrit ≦25 | at either 14 or |
| | 14 dpc/Total | at 21 dpc/Total | 21 dpc/Total |
| Group | No. Evaluated | No. Evaluated | No. Evaluated |
| Negative | 0/23 | 0/21 | 0/25 |
| Control | | | |
| Positive | 27/38 | 15/28 | 32/39 |
| Control | (71.1%) | (53.6%) | (82.1%) |
| Progeny from | 4/36 | 1/35 | 5/38 |
| CIAV | | | |
| Vaccinated Hens | (11.1%) | (2.9%) | (13.2%) |
| Fisher's Exact | <0.001^{a} | <0.001^{a} | <0.001^{a} |
| Test p = | | | |

^{a}Statistical difference by Fisher's Exact Test at p < 0.001 between positive controls and progeny from CIAV vaccinated breeder chickens.

TABLE 37
Study 2 Summary of Clinical Signs and Mortality of
CIAV Challenged Chicks from 37-week-old
Vaccinated and Non-vaccinated Breeder Chickens.

| | No. with | | No. with Clinical |
|---|---|---|---|
| | Clinical | | Signs or |
| Group | Signs/Total | No. Dead/Total | Dead/Total |
| Negative Control | 0/25 | 0/25 | 0/25 |
| Positive Control | 6/40 (15%) | 12/40 (30%) | 16/40 (40.0%) |
| Progeny from CIAV | 0/40 | 3/40 (7.5%) | 3/40 (7.5%) |
| Vaccinated Hens | | | |
| Fisher's Exact Test p = | 0.013^{a} | 0.010^{a} | <0.001^{a} |

^{a}Statistical difference by Fisher's Exact Test at p < 0.05 between positive control group and progeny from CIAV vaccinated breeder chickens.

TABLE 38
Study 2 Summary of CIA Gross Lesion Scores of CIAV
Challenged Progeny from 37-Week-old CIAV Vaccinated
and Non-vaccinated Breeder Chickens.

| | No. Birds with Gross Lesion Scores | | | | |
|---|---|---|---|---|---|
| | (GLS) ≧1/Total No. Birds at | | | | No. |
| | Post-mortem | | | | Birds with |
| | | Bone | | | GLS |
| Group | Liver | Marrow | Thymus | Muscle | ≧1/Total |
| Negative | 0/25 | 0/25 | 0/25 | 0/25 | 0/25 |
| Control | | | | | |
| Positive | 0/40 | 22/40 (55%) | 22/40 (55%) | 0/40 | 24/40 (60%) |
| Control | | | | | |
| Progeny | 0/40 | 1/40 (2.5%) | 3/40 (7.5%) | 0/40 | 3/40 (7.5%) |
| from CIAV | | | | | |
| Vaccinated | | | | | |
| Hens | | | | | |
| Mann- | 0.500 | <0.001^{a} | <0.001^{a} | 0.293 | <0.001^{a} |
| Whitney | | | | | |
| Test p = | | | | | |
| Fisher's | NA^{b} | NA | NA | NA | <0.001^{c} |
| Exact | | | | | |
| Test p = | | | | | |

^{a}Statistical difference at p < 0.001 between positive control group and progeny from CIAV vaccinated hens by Mann-Whitney Test.
^{b}Not applicable.
^{c}Statistical difference at p < 0.001 between positive control group and progeny from CIAV vaccinated hens by Fisher's Exact Test.

### Example 8

Evaluation of Tumorigenicity in Chickens Following Various Treatments on MDCC-MSB-1 Cells to Inactivate Marek's Virus A tumorigenicity study was conducted on the MDCC-MSB-1 cell line substrate used for propagation of the Del-Ros strain of CIAV. The objective of this study was to demonstrate that a cell-free supernatant fluid derived from actively growing cell cultures lack the ability to induce Marek's Disease (MD) tumors when inoculated into susceptible chickens.

Groups of 25 to 36, SPF white leghorns chicks, aged 1-5 days were inoculated with various inocula as shown in Table 39.

Chicks of both trials were observed daily for clinical signs of MD, and the dead birds were necropsied and examined for gross lesions of MD during an 8 week observation period. At the end of the observation period, all of the remaining birds (including the negative controls) were sacrificed with C0₂ and examined for MD related gross lesions. Samples of questionable or suspicious lesions were collected in 10% formaldehyde solution for histopathological examination.

The MSB-1 cells without an additional processing step at a dose of 1×10⁶ viable cells induced tumors in 2 of 36 chickens. However, additionally processed cell free media did not induce tumors in chickens. The results are summarized in Table 40.

The data obtained from this study indicate that if MSB-1 cells are used as the substrate for virus production such as for CIAV, it is necessary to remove MSB-1 cells from the harvested virus to prevent the potential of Marek's disease in chickens receiving the CIAV vaccine. Removal of the cells can be accomplished by filtering the MSB-1 virus infected cells through a coarse filter (5 u size Millipore) to remove the cells. The cell-free virus fluid would be safe for to administer to chickens.

The results of this study demonstrated that additional processing steps of the live virus (i.e., natural sedimentation followed by filtration through 5 u Millipore filter) of the MSB-1 cells eliminates the possibility of a vaccine produced in this cell line from inducing any MD related tumors in chickens.

The results suggest that filtration of the supernatant fluid of chicken anemia virus produced in MSB-1 cells will prevent the associated risk of MD tumor formation when administered to chickens.

TABLE 39
Experimental design for the MSB-1 in-vivo tumorigenicity test:

| | | | | Dose of |
|---|---|---|---|---|
| Group | | Total no. of | Route of | Inoculum/ |
| No. | Treatments | chicks | Inoculation | Chick |
| 1. | ~1.0 × 10⁶ viable MSB-1 cells | 36 | SQa | 0.2 ml |
| | grown in RPMI 1640 medium | | | |
| | supplemented with FBS. | | | |
| 2. | Supernatant from a centrifuged | 35 | SQ | 0.2 ml |
| | (2000 rpm for 10 min.) MSB-1 | | | |
| | cell suspension. | | | |
| 3. | RPMI 1640 medium | 25 | SQ | 0.2 ml |
| | supplemented with FBS | | | |
| | (Medium control). | | | |
| 4. | 3.0 × 10⁵ viable MSB-1 cells/ml, | 35 | SQ | 0.2 ml |
| | allowed to sediment naturally | | | |
| | for overnight and the resulting | | | |
| | supernatant then filtered through | | | |
| | 5 u Millipore filter, and finally treated | | | |
| | at 41° C. for 24 hours before used | | | |
| | for chick inoculation. | | | |
| 5. | 3.0 × 10⁵ viable MSB-1 cells/ml, | 35 | SQ | 0.2 ml |
| | allowed to sediment naturally for | | | |
| | overnight and the resulting | | | |
| | supernatant then filtered through | | | |
| | 5 u Millipore filter before | | | |
| | using for chick inoculation. | | | |
| 6. | 3.0 × 10⁵ viable cells/ml, freeze | 35 | SQ | 0.2 ml |
| | and thawed 3 times at −20° C. | | | |
| | and then centrifuged at 2000 rpm | | | |
| | for 15 min., the resulting supernatant | | | |
| | then filtered through 5 u Millipore | | | |
| | filter, and lastly the filtrate was | | | |
| | exposed to 41° C. for 24 hours | | | |
| | before using for chick inoculation. | | | |
| 7. — | Negative controls | 35 | ND | ND |

^{a}Subcutaneous

TABLE 40
Tumorigenicity test results of MDCC-MSB-1 cells.

| | | Necropsy results | | | | |
|---|---|---|---|---|---|---|
| | | (MD related | | Total Pos. | % | |
| Test | Total | lesions) | | For MD | MD | |
| groups | mortality | Gross | Histopath | lesion | Pos. | Remarks |
| 1 | 2/36 | 7 | 4 | 11 | 30.5 | 1 × 10⁶ viable cells/chick |
| | | | | | | indicates risk of MD tumor |
| | | | | | | formation |
| 2 | 0/35 | 2 | 3 | 5 | 14.3 | Centrifuging at 2000 rpm |
| | | | | | | for 15 min. is not enough |
| | | | | | | to eliminate cells from the |
| | | | | | | cell suspension, resulting |
| | | | | | | in low incidence of tumor |
| | | | | | | formation. |
| 3 | 0/25 | 0 | ND | 0 | 0.0 | The medium used for |
| | | | | | | growing MSB-1 cells is |
| | | | | | | safe for use |
| 4 | 1/35 | 0 | ND | 0 | 0.0 | Cell free filtrate does not |
| | | | | | | induce tumor; safe for use |
| | | | | | | in vaccine production |
| 5 | 0/35 | 0 | ND | 0 | 0.0 | Cell free filtrate does not |
| | | | | | | induce tumor; safe for use |
| | | | | | | in vaccine production |
| 6 | 0/35 | 0 | ND | 0 | 0.0 | Cell free filtrate does not |
| | | | | | | induce tumor; safe for use |
| | | | | | | in vaccine production |
| 7 | 0/35 | 0 | ND | 0 | 0.0 | No tumors in the negative |
| | | | | | | controls |

### Example 9

### The Effects of Freeze-Thaw and 37° C. Incubation on the Viability of Marek's Disease Virus

Freeze-thaw up to 3 cycles could not completely inactivate Marek's disease virus (MDV) in tissue culture medium, but reduced the number of plaques significantly. However, following 3 freeze-thaws and then 3 days' incubation at 37° C., there was no MDV serotype 1 virus detected by IFA.

Marek's disease virus and turkey herpesvirus (HVT) exist in either cell-associated or cell free states, which have greatly different survival properties. The infectivity of cell-associated virus stock is directly related to viability of the cells containing the virus. The infectivity of cell free virus preparation was reported to be sensitive to different pH and temperatures. The viability of MDV, Rispen's strain, under freeze-thaw and 37° C. incubation treatments was investigated.

### Materials and Methods

Cells: The CEF cells (primary CEF in roller bottle, secondary CEF in 60 mm tissue culture plates) were prepared from 9 to 11 days-old SPF chicken embryos (SPAFAS).

Virus: The effect of freeze-thaw on the viability of Rispen's virus was investigated by conducting an inactivation (kill) study. The active Rispen's infected CEF cells were harvested at 43 hpi. The infected cells were resuspended in minimal essential medium (MEM) supplemented with fetal and calf sera and tryptose phosphate broth, and filled into 20 tubes. The concentration of the cells was 36×10⁶ cells per ml. Samples were treated by freezing at −70° C. followed by thawing at room temperature, from one up to three cycles, then incubated at 37° C., from one up to 15 days. The samples, with or without dilution, were inoculated into secondary CEF monolayer in 60 mm tissue culture plates in duplicate, and incubated at 37° C. for 4-5 days. Titers were scored by count plaques under a microscope with and without IFA stain with MDV serotype 1-specific monoclonal antibody 2BN90.

### Results

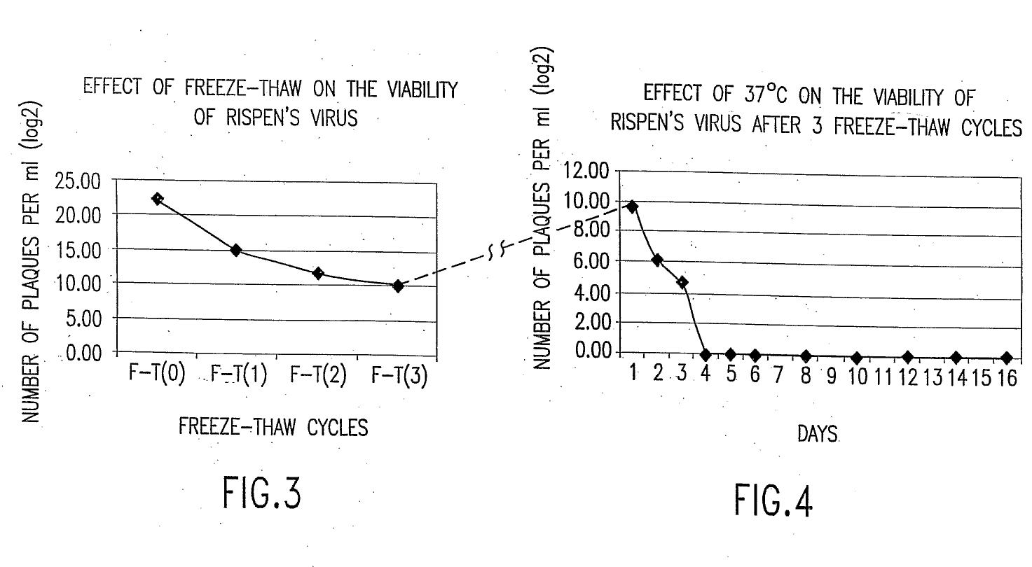

The MDV plaques were counted and reported as the average plaque forming unit (pfu) per ml. The results indicated that up to 3 freeze-thaw cycles did not completely inactivate MDV Rispeen's strain in tissue culture medium, but the number of plaques that indicated evidence of viable virus were reduced significantly. However, with 3 or more days incubation at 37° C. after 3 freeze-thaw cycles, there were no plaques detected by IFA (Table 41, and FIGS. 3 and 4), suggesting that combining 3 freeze-thaw cycles with a 3-day incubation at 37° C. can completely destroy MDV infectivity in the cell free medium.

TABLE 41
The average MDV plaques resulting following each treatment

| Treatment | Results |
|---|---|
| Initial titer prior to freeze-thaw: | 5.4 × 10⁶ pfu/ml |
| Freeze-thaw once: | 3 × 10⁴ pfu/ml |
| Freeze-thaw twice: | 3 × 10³ pfu/ml (By IFA) |
| Freeze-thaw 3 times: | 800 pfu/ml (By IFA) |
| Freeze-thaw 3 times + 37° C. 1 day: | 70 pfu/ml (By IFA) |
| Freeze-thaw 3 times + 37° C. 2 day: | 25 pfu/ml (By IFA) |
| Freeze-thaw 3 times + 37° C. 3 day: | 0 |
| Freeze-thaw 3 times + 37° C. 4 day: | 0 |
| Freeze-thaw 3 times + 37° C. 5 day: | 0 |
| Freeze-thaw 3 times + 37° C. 7 day: | 0 |
| Freeze-thaw 3 times + 37° C. 9 day: | 0 |
| Freeze-thaw 3 times + 37° C. 11 day: | 0 |
| Freeze-thaw 3 times + 37° C. 13 day: | 0 |
| Freeze-thaw 3 times + 37° C. 15 day: | 0 |

### Example 10

### Comparison of Sequences for CIAV Strains

There are numerous reported strains of CIAV. Some of these have been sequenced and their sequences deposited. A chair comparing the amino acid sequence of several of the known strains is provided In Table 42. It is based on a pile up of sequences obtained from the NCBI database.

TABLE 42
Specific Amino Acid Changes in VP1, VP2 and VP3 of Several CAV Isolates

| | | VPI Amino Acid Position | | | | | | | | | | | |
|---|---|---|---|---|---|---|---|---|---|---|---|---|---|
| Isolate | Identification | 14 | 84 | 92 | 144 | 157 | 229 | 251 | 254 | 287 | 370 | 413 | 447 |
| DRP5 | 5^{th} embryo | A | V | G | E | V | S | R | E | S | G | S | G |
| | passage Del- | | | | | | | | | | | | |
| | Ros strain | | | | | | | | | | | | |
| DR | Del-Ros strain | A | L | G | E | V | S | R | E | S | G | S | G |
| TX | Texas Isolate | A | L | G | E | V | F | R | E | T | G | S | T |
| Cux-1 | Cuxhaven-1 | S | L | G | D | V | S | Q | G | A | S | A | T |
| IV | Intervet | A | L | D | E | M | S | R | G | T | S | A | T |
| | Vaccine | | | | | | | | | | | | |

| | | VP2 Amino | | | | | | | |
|---|---|---|---|---|---|---|---|---|---|
| | | Acid Position | | VP3 Amino Acid Position | | | | | |
| Isolate | Identification | 153 | 169 | 4 | 23 | 73 | 103 | 116 | 118 |
| DRP5 | 5th embryo | V | D | L | R | V | S | R | C |
| | passage | | | | | | | | |
| | Del-Ros strain | | | | | | | | |
| DR | Del-Ros strain | V | D | L | R | V | S | R | C |
| TX | Texas Isolate | V | D | L | R | V | S | R | C |
| Cux-1 | Cuxhaven-1 | A | D | L | R | V | S | K | R |
| IV | Intervet Vaccine | V | G | P | Q | A | N | R | C |

Nucleotide and amino acid sequences for the Del Ros strain are provided in the Sequence listing and also at NCBI accession no. AF313470. Nucleotide and amino acid sequences for additional other strains of CIAV can be found as follows: intervet-NCBI accession no. D100068; Cuxhaven-1-NCBI accession no. NC001427; and CAV-15-NCBI accession no. AF372658. A nucleotide by nucleotide or amino acid by amino acid comparison of these and other sequence can be routinely made.

### Example 11

### Use of a Live Chicken Infectious Anemia Virus Vaccine Improves Broiler Flock Weight Performance Without Adverse Safety Concerns

Evaluation and Results: Live chicken anemia virus vaccine was used in broilers at 18 days of age on a farm of 119,800 meat-type chickens. This farm had been experiencing a downward trend in performance, as measured by the rate of condemned pounds of meat due to health related issues. Chickens were vaccinated in the drinking water at 18 days of age, and the flock of 113,453 chickens was sent to the processing plant at 41 days of age. Livability, bird weight and number of pounds of meat condemned by the inspector were evaluated against flocks of similar age processed the same week. The term “condemned,” as used throughout, means a poultry inspector judged the meat not fit for human consumption and not wholesome. The flock performance results are found in Table 43.

TABLE 43
Flock Performance Results

| | No. | | | | | |
|---|---|---|---|---|---|---|
| | Birds | Age in | % | (Lbs.) Ave. | Total Lbs. | % Lbs. |
| Group | Sold | Days | Livability | Wt. | Condemned | Condemned |
| Test Farm | 113,453 | 41 | 94.70 | 4.199 | 3673 | 0.765 |
| (Vaccinated) | | | | | | |
| Other Farms | 351,325 | 41, 42, | 94.62 | 4.075 | 5151 | 0.360 |
| (Not | | 43 | | | | |
| Vaccinated) | | | | | | |

Livability of the vaccinated chickens was better than the non-vaccinated chickens and also demonstrated that the vaccine was safe for use. The difference in weight of the 0.124 pounds was highly significant because it demonstrated that the vaccinates had a reduced time to market by achieving higher weight in less time. There was not an improvement in % pounds condemned compared to other flocks being processed that week, but there was a significant improvement compared to previous flocks on this same farm as detailed in Example 12.

### Example 12

### Use of Live Chicken Infectious Anemia Vaccine to Reduce Health Related Condemnations on a Broiler Farm.

Evaluation and Results: The farm, as described in Example 11, had a past history of high rates of condemned pounds of meat due to health related issues. A comparison was made on the incidence of pounds of condemned meat when the flocks reared on this farm were processed with and without use of live chicken infectious anemia virus vaccine. The results are found in Table 44.

TABLE 44
Comparison of Lbs. of Condemned Meat

| Flock Vaccine | | | Total Pounds | % pounds |
|---|---|---|---|---|
| Treatment | No. Birds Sold | Age | Condemned | Condemned |
| 100% | 113,453 | 41 | 3673 | 0.765 |
| Vaccinated | | | | |
| 50% | 112,475 | 38 | 4532 | 1.033 |
| Vaccinated | | | | |
| None | 112,929 | 40 | 5787 | 1.215 |
| Vaccinated | | | | |

The results showed that as chicken infectious anemia vaccine was used on this farm there were less condemned pounds of meat due to health related issues even though the 100% vaccinated flock was grown in the winter months when disease problems are the highest.

### Example 13

### A Reduction in Pounds of Meat Condemned when Chicken Infectious Anemia Vaccine is Used.

Evaluation and Results: A farm with a capacity of 48,000 broilers exhibited same poor performance as the farm described in Example 11. Performance data at processing were evaluated for three consecutive flocks, as seen in Table 45.

TABLE 45
Performance Data at Processing

| | | | | | | % Lbs. |
|---|---|---|---|---|---|---|
| Flock | No. Of | | | Lbs. | Total | Con- |
| Vaccine | Birds | | % Liva- | Average | Pounds | demned |
| Treatment | Sold | Age | bility | Weight | Condemned | in Flock |
| 50% | 45,594 | 40 | 94.99 | 4.152 | 1,117 | 0.567 |
| Vaccinated | | | | | | |
| None | 43,630 | 39 | 90.90 | 4.069 | 6,575 | 3.571 |
| Vaccinated | | | | | | |
| None | 43,787 | 39 | 91.61 | 4.048 | 6,670 | 3.627 |
| Vaccinated | | | | | | |

Of three consecutive flocks at this farm, the flock that was 50% vaccinated was grown in the most severe disease period of winter had superior growing performance. Performance benefits were realized in significantly increased livability and reduced pounds of condemned meat which is directly correlated with decreased disease problems compared to prior non-vaccinated flocks on the same farm.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A chicken infectious anemia virus (CIAV) vaccine, comprising live CIAV passaged in MDCC-MSB-I (MSB-I) cells, wherein the vaccine does not cause Marek's Disease.

2. The CIAV vaccine of claim 1, wherein the vaccine does not produce gross lesions in chicken embryos.

3. The CIAV vaccine of claim 1, wherein the vaccine does not produce anemia in chicken embryos.

4. The CIAV vaccine of claim 1, wherein the vaccine can be safely administered to chickens less than 28 days of age.

5. The CIAV vaccine of claim 1, wherein the vaccine can be safely administered to chickens greater than 28 days of age.

6. The CIAV vaccine of claim 1, wherein the vaccine can be safely administered to chicken embryos in ovo.

7. The CIAV vaccine of claim 6, wherein the chicken embryos are at between 16 and 20 days of incubation.

8. The CIAV vaccine of claim 1, wherein the vaccine is inactivated.

9. A method of making a CIAV vaccine, comprising culturing CIAV in MSB-I cells, and removing or killing any Marek's disease virus present in the CIAV-containing MSB-I cell culture.

10. The method of claim 9, comprising subjecting the CIAV-containing MSB-I cell culture to at least 3 cycles of freezing and thawing, followed by a step of maintaining the cells for about 3 days at about 37° C.

11. The method of claim 9, comprising the step of filtering the MSB-I cell culture through a 5 micron filter.

12. The method of claim 10, wherein the method makes a vaccine that does not cause Marek's disease in chickens immunized with the vaccine.

13. A method of immunizing a chicken against CIAV infection, comprising administering to the chicken an amount of the CIAV vaccine of claim 1 sufficient to induce an immune response to CIAV.

14. The method of claim 13, wherein the immune response is protective against infection by CIAV.

15. The method of claim 13, wherein the immune response is protective against clinical disease caused by CIAV infection.

16. The method of claim 13, wherein the immune response produces antibodies that are protective against CIAV infection in the progeny of immunized chickens.

17. The method of claim 13, wherein the vaccine is administered to chickens from about 1 to 12 weeks of age.

18. The method of claim 9, wherein the vaccine can be administered in combination with Marek's disease vaccine, infectious bursal disease vaccine, reo virus vaccine, Newcastle disease vaccine, infectious bronchitis disease vaccine, pneumovirus vaccine and avian influenza virus vaccine.

19. The method of claim 13, wherein the vaccine is administered in drinking water.

20. The method of claim 13, wherein the vaccine is administered parenterally.

21. The method of claim 20, wherein the vaccine is administered by spray 22.

22. The method of claim 20, wherein the vaccine is administered by injection.

23. The method of claim 11, wherein the method makes a vaccine that does not cause Marek's disease in chickens immunized with the vaccine.
